(19) United States
(12) Patent Application Publication
(10) Pub. No.: US 2023/0073428 A1
(43) Pub. Date: Mar. 9, 2023

(54) METHODS OF TREATING AND ASSESSING PULMONARY ARTERIAL HYPERTENSION WITH SELEXIPAG

(71) Applicant: ACTELION PHARMACEUTICALS LTD, Allschwil (CH)

(72) Inventors: Emmanuelle COTTREEL, Allschwil (CH); Loïc PERCHENET, Allschwil (CH)

(21) Appl. No.: 17/759,467

(22) PCT Filed: Feb. 2, 2021
(86) PCT No.: PCT/EP2021/052374

§ 371 Date: Jul. 26, 2022

Related U.S. Application Data
(60) Provisional application No. 62/969,366, filed on Feb. 3, 2020.

(51) Int. Cl. A61K 31/4965; A61P 9/12; A61B 5/00

(52) U.S. Cl. CPC A61K 31/4965; A61P 9/12; A61B 5/4836

(57) ABSTRACT

The present disclosure provides methods for treating pulmonary arterial hypertension in a patient in need thereof, comprising (a) performing magnetic resonance imaging (MRI) on the right ventricle of the patient to provide a MRI baseline image; (b) administering a therapeutically effective amount of selexipag; (c) performing MRI on the right ventricle of the patient to provide a MRI test image; and (d) comparing the MRI baseline image with the MRI test image.

### CROSS-REFERENCE TO RELATED APPLICATIONS

This application claims the benefit of U.S. Provisional Patent Application No. 62/969,366, filed Feb. 3, 2020, the disclosure of which is incorporated by reference herein.

### BACKGROUND

Pulmonary arterial hypertension (PAH) is a serious chronic disorder of the pulmonary circulation, a syndrome of diverse etiology and pathogenesis characterized by a progressive increase in pulmonary arterial pressure (PAP) and in pulmonary vascular resistance (PVR) potentially leading to right heart failure and death.

PAH is associated with structural changes in both pulmonary vasculature and the right ventricle (RV). The changes in vascular structure involve three combined elements: vasoconstriction, vascular-wall remodeling, and thrombosis in situ. These changes to the pulmonary vessels result in reduced pulmonary artery compliance, thereby resulting in a stiffening of the vessels. The changes in the RV mainly consist of hypertrophy, dilation, altered contractility, and septal bowing. Collectively, these changes of the RV are termed remodeling. However, imaging the RV is complex and difficult to delineate.

PAH is hemodynamically defined as a resting mean pulmonary arterial pressure (mPAP) equal to or greater than 25 mmHg with normal pulmonary arterial wedge pressure (PAWP) or left ventricular end-diastolic pressure (LVEDP) (≤15 mmHg) and a PVR greater than 3 Wood units (WU). See, Hoeper, Definitions and diagnosis of pulmonary hypertension. J. Am. Coll. Cardiol. 2013; 62:D42-50. The updated clinical classification of pulmonary hypertension classifies the numerous conditions that are known to lead or be associated with the development of PAH based on their similar clinical presentation, pathology, pathophysiology, prognosis, and, most of all, similar therapeutic approach.

Selexipag (ACT-293987/JNJ-67896049) is a selective, orally available, long-acting, non-prostanoid agonist of the prostacyclin receptor, approved and commercially available for the treatment of patients with PAH in the United States, the European Union, Japan, and other countries. To date, cardiac imaging data on selexipag are lacking even in view of RV function being a determinant of survival. For example, in order to compensate for the increased pulmonary vascular resistance, the RV remodels. It dilates and hypertrophies, but may be unable to maintain a sufficient cardiac output. There is a need to evaluate the effect of selexipag on the right ventricle, and, in particular, evaluate such effects by means of magnetic resonance imaging (MRI) to assess cardiac changes related to treatment and/or to make adjustments thereto.

### SUMMARY

The present disclosure provides methods for treating pulmonary arterial hypertension using magnetic resonance imaging (MRI). In some embodiments, the present disclosure provides methods for treating pulmonary arterial hypertension in a patient in need thereof, comprising (a) performing MRI on the right ventricle of the patient to provide a MRI baseline image; (b) administering a therapeutically effective amount of selexipag; (c) performing MRI on the right ventricle of the patient to provide a MRI test image; and (d) comparing the MRI baseline image with the MRI test image. In some embodiments, step (d) is performed at least about 26 weeks after initiating the administration of the selexipag. In other embodiments, step (d) is performed at least about 52 weeks after initiating the administration of the selexipag. Analysis of a first comparison and a subsequent second comparison may be used to measure sustainability of the treatment. For example, step (d) may be performed at about 26 weeks and at about 52 weeks. The results of such methods are considered in connection with, for example, adjustments to a patient's treatment.

### BRIEF DESCRIPTION OF THE DRAWINGS

FIG. 1 is a schematic showing a study design to assess effects of selexipag on RV function.

### DETAILED DESCRIPTION OF ILLUSTRATIVE EMBODIMENTS

In the present disclosure the singular forms “a”, “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about” or “substantially” it will be understood that the particular value forms another embodiment. In general, use of the term “about” or “substantially” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about” or “substantially”. In other cases, the gradations used in a series of values may be used to determine the intended range available to the term “about” or “substantially” for each value. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiments and such a combination is considered to be another embodiment. Conversely, various features of the invention that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

### Methods

The present disclosure provides methods of treating pulmonary arterial hypertension in a patient in need thereof with selexipag by utilizing magnetic resonance imaging. Because MRI is a noninvasive technique, it is desirably used to monitor PAH patients receiving treatment with selexipag, over more invasive techniques such as right heart catherization.

The terms “pulmonary arterial hypertension” and “PAH” are interchangeable and define a condition of pulmonary hypertension where the patient has high blood pressure in the lungs. PAH occurs when the very small arteries throughout the lungs narrow in diameter, which increases the resistance to blood flow through the lungs resulting in a higher afterload for the right ventricle. In some embodiments, the underlying cause of the narrowing is not known, i.e., idiopathic pulmonary hypertension. PAH also is classified into subgroups including (i) familial, or heritable PAH, (ii) PAH caused by drugs or toxins, (iii) PAH associated connective tissue disease (such as scleroderma or lupus), congenital heart disease (optionally with simple systemic-to-pulmonary shunt at least 1 year after surgical repair), high blood pressure in the liver, HIV and infections (schistosomiasis), (iv) PAH caused by rare blood conditions (pulmonary veno-occlusive disease or pulmonary capillary hemangiomatosis, or (v) PAH in babies (persistent pulmonary hypertension of the newborn).

The methods described herein also may include a determination that the patient has PAH. Typically, that determination is made by an attending physician. A diagnosis or determination of PAH may be performed using techniques known by those of skill in the art. For example, a right-heart catheterization may be conducted to confirm pulmonary arterial hypertension in a patient.

Severity of PAH in a patient is generally evaluated by a classification system, i.e., the World Health Organization (WHO) class system. See, Table A.

TABLE A
WHO Class System for PAH Patients

| Class | Patient's Symptoms |
|---|---|
| I | No limitations on physical activity. |
| | Ordinary physical activity does not cause |
| | undue dyspnea of fatigue, chest pain, or near |
| | syncope. |
| II | Slight limitation of physical activity. |
| | Comfortable at rest. |
| | Ordinary physical activity causes undue |
| | dyspnea or fatigue, chest pain, or near syncope. |
| III | Marked limitation of physical activity. |
| | Comfortable at rest. |
| | Less than ordinary activity causes undue |
| | dyspnea or fatigue, chest pain or near syncope. |
| IV | Inability to carry out any physical activity |
| | without symptoms. |
| | Manifest signs of right heart failure. |
| | Dyspnea and/or fatigue may be present at rest. |
| | Discomfort is increased by any physical activity. |

In general, a higher PAH class indicates a more severe disease state and/or greater urgency for a patient to be accurately diagnosed and started on PAH therapy. Thus, the methods reduce the risk of a patient progressing from a lower WHO PAH class to a higher WHO PAH class. In some embodiments, the methods reduce the risk of a PAH patient progressing from a WHO class I to a WHO class II, WHO class I to a WHO class III, WHO class I to a WHO class IV, WHO class II to a WHO class III, WHO class II to a WHO class IV, or WHO class III to a WHO class IV.

The present disclosure provides methods for treating pulmonary arterial hypertension in a patient in need thereof, comprising the use of magnetic resonance imaging (MRI). Not only does MRI provide a means for monitoring the patient's condition or progression of PAH, but it provides an effective tool in assessing the need to, for example, adjust the selexipag dose. The term “magnetic resonance imaging” or “MRI” as used herein refers to a non-invasive imaging technology that produces three dimensional detailed anatomical images. MRI may be utilized for a variety of purposes including, but not limited to, imaging the heart and, thereby, measuring a variety of parameters associated with heart function. This often is referred to as “cardiac MRI” or “pulmonary artery flow MRI”. Cardiac magnetic resonance imaging or “CMRI” can provide information about different aspects of the heart such as, without limitation, the right and left ventricles, preferably the right ventricle. In some embodiments, MRI is utilized to measure one or more of right ventricular end diastolic volume (RVEDV), right ventricular ejection fraction (RVEF), right ventricular end systolic volume (RVESV), right ventricular global longitudinal strain (RVGLS), right ventricular stroke volume (RVSV), right ventricular mass, and right main PA pulsatility.

The particular type of MRI technique utilized may selected by one skilled in the art and includes spin echo, gradient echo, inversion recover, and magnetic resonance angiography and venography. One of skill in the art also would readily be able to select a suitable MRI instrument for use in the methods described herein. The MRI instrument may be open or closed (such as a Tesla MRI), although closed is preferred. Contrast agents may be utilized as determined by those skilled in the art, but are not required.

Additional imaging techniques may be utilized to complement the MRI. In some embodiments, the additional imaging technique include, without limitation, ultrasound, echocardiography, cardiac computerized tomography (CT), and/or nuclear medicine. Non-imaging techniques also are optionally used to complement the MRI. These include, without limitation, bloodwork, physical exertion tests, electrocardiograms, pathology specimens, among others.

The methods described herein include imaging or performing MRI on the patient before selexipag treatment is initiated. In some embodiments, the right ventricle of the heart is imaged before selexipag treatment is initiated. By doing so, baseline MRI images are obtained, which can later be compared with images that are acquired after selexipag treatment. The images may also be used to determine one or more baseline cardiac measurements, such as a baseline RVEDV, baseline RVEF, baseline RVESV, baseline RVGLS, and/or baseline RVSV, among others.

As known in the art, RVESV is the volume of blood in the right ventricle at the end of contraction and at the beginning of filling; RVESV is the lowest volume of blood in the ventricle. Patients with PAH may have higher values of RVESV. Thus, measuring the effects of selexipag on PAH by measuring RVESV provides the clinician with a real-time method of determining if treatment is successful and/or needs to be adjusted. The methods disclosed herein are effective in improving, i.e., lowering the RVESV from the baseline RVESV, as measured by MRI, after, for example, about 26 weeks, and/or after, for example, about 52 weeks following selexipag initiation.

RVEDV is the amount of blood that is in the right ventricle right before the heart contracts. Patients with PAH may have higher values of RVEDV. The methods disclosed herein are effective in improving the baseline RVEDV, as measured by MRI, after, for example, about 26 weeks, and/or after, for example, about 52 weeks following selexipag initiation. In some embodiments, the methods decrease the baseline RVEDV

RVSV is the quantity of blood that the heart pumps out of the right ventricle with each beat. RVSV is calculated as follows:

RVSV=RVEDV−RVESV

Patients with PAH may have low values of RVSV. The methods disclosed herein are effective in improving the baseline RVSV, as measured by MRI, after, for example, about 26 weeks, and/or after, for example, about 52 weeks following selexipag initiation. In some embodiments, the methods increase the baseline RVSV. In other embodiments, the methods increase the patient's RVSV by at least about 5 mL, based on the baseline RVSV. In further embodiments, the methods increase the patient's RVSV by about 5 to about 15 mL. In yet other embodiments, the methods increase the patient's RVSV by about 8 to about 12 mL. In still further embodiments, the methods increase the patient's RVSV by about 8 mL.

RVEF is the measurement of how much blood (%) is pumped out of the right side of the heart to the lungs for oxygen. RVEF may be calculated as follows:

$$RVEF = \frac{RVSV}{RVEDV} \times 100$$

Patients with PAH may have low values of RVEF. The methods disclosed herein are effective in improving the baseline RVEF, as measured by MRI, after, for example, about 26 weeks, and/or after, for example, about 52 weeks following selexipag initiation. The methods disclosed herein are effective in raising the RVEF. In some embodiments, the RVEF is raised, for example, by at least about 10%, based on the baseline RVEF.

RVGLS is a measure of the percent change in myocardial length from a relaxed state to the contractile state in the right ventricle. Patients with PAH may have larger negative values of RVGLS. The methods disclosed herein are effective in improving the baseline RVGLS, as measured by MRI, after, for example, about 26 weeks, and/or after, for example, about 52 weeks following selexipag initiation. The methods disclosed herein are effective in improving the RVGLS. In some embodiments, the RVGLS is improved, for example, by at least about 10%, based on the baseline RVGLS.

RV mass is a measure of the muscle mass of the right ventricle of the heart. Patients with PAH may have a higher RV mass. The methods disclosed herein are effective in improving the baseline RV mass, as measured by MRI, after about 12 months following selexipag initiation. The methods disclosed herein are effective in lowering the RV mass. In some embodiments, the RV mass is lowered, for example, by at least about 10%, based on the baseline RV mass.

Patients with PAH may have lower values of right main PA pulsatility. The methods disclosed herein are effective in improving the baseline right main PA pulsatility, as measured by MRI, after, for example, about 26 weeks, and/or after, for example, about 52 weeks following selexipag initiation. The methods disclosed herein are effective in raising the right main PA pulsatility. In some embodiments, the right main PA pulsatility is raised, for example, by at least about 10%, based on the baseline right main PA pulsatility.

After baseline values have been acquired, PAH treatment with selexipag is initiated by administering a therapeutically effective amount of selexipag. Typically, selexipag is administered at a starting dose and is increased to determine an individual maximum tolerated dose (iMTD) during a dose adjustment phase, and the selexipag dosage maintained during a maintenance phase. The iMTD and maintenance dose as used herein refers to the amount of selexipag that may be administered to a patient per day based on tolerability. Thus, the iMTD and maintenance dose are typically evaluated for each patient on an individual basis. In some aspects, the starting dose of selexipag is the same as the iMTD and/or maintenance dose. In further aspects, the starting dose of selexipag is lower than the iMTD and/or maintenance dose.

The selexipag starting dose, iMTD, and/or maintenance dose, on a daily basis, is at least about 10 μg. In some embodiments, the selexipag starting dose, iMTD, and/or maintenance dose, on a daily basis, is at least about 100, about 200, about 300, about 400, about 500, about 600, about 700, about 800, about 900, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, about 3000, about 3100, about 3200, about 3300, about 3400, or about 3500 μg. In some embodiments, the selexipag starting dose is about 200 μg, twice daily. The daily dose may be administered once daily, twice daily, or thrice daily, preferably twice daily.

Desirably, the selexipag iMTD and maintenance doses do not exceed about 1600 μg twice daily, i.e., 3200 μg per day. In some embodiments, the selexipag iMTD and/or maintenance dose, twice daily, is about 100 to about 3500 μg, about 200 to about 3200, about 200 to about 3000, about 200 to about 2800, about 200 to about 2600, about 200 to about 2400, about 200 to about 2200, about 200 to about 2000, about 200 to about 1800, about 200 to about 1600, about 200 to about 1400, about 200 to about 1200, about 200 to about 1000, about 200 to about 800, about 200 to about 600, about 200 to about 400, about 400 to about 3200, about 400 to about 3000, about 400 to about 2800, about 400 to about 2600, about 400 to about 2400, about 400 to about 2200, about 400 to about 2000, about 400 to about 1800, about 400 to about 1600, about 400 to about 1400, about 400 to about 1200, about 400 to about 1000, about 400 to about 800, about 400 to about 600, about 600 to about 3200, about 600 to about 3000, about 600 to about 2800, about 600 to about 2600, about 600 to about 2400, about 600 to about 2200, about 600 to about 2000, about 600 to about 1800, about 600 to about 1600, about 600 to about 1400, about 600 to about 1200, about 600 to about 1000, about 600 to about 800, about 800 to about 3200, about 800 to about 3000, about 800 to about 2800, about 800 to about 2600, about 800 to about 2400, about 800 to about 2200, about 800 to about 2000, about 800 to about 1800, about 800 to about 1600, about 800 to about 1400, about 800 to about 1200, about 800 to about 1000, about 1000 to about 3200, about 1000 to about 3000, about 1000 to about 2800, about 1000 to about 2600, about 1000 to about 2400, about 1000 to about 2200, about 1000 to about 2000, about 1000 to about 1800, about 1000 to about 1600, about 1000 to about 1400, about 1000 to about 1200, about 1200 to about 3200, about 1200 to about 3000, about 1200 to about 2800, about 1200 to about 2600, about 1200 to about 2400, about 1200 to about 2200, about 1200 to about 2000, about 1200 to about 1800, about 1200 to about 1600, about 1200 to about 1400, about 1400 to about 3200, about 1400 to about 3000, about 1400 to about 2800, about 1400 to about 2600, about 1400 to about 2400, about 1400 to about 2200, about 1400 to about 2000, about 1400 to about 1800, about 1400 to about 1600, about 1600 to about 3200, about 1600 to about 3000, about 1600 to about 2800, about 1600 to about 2600, about 1600 to about 2400, about 1600 to about 2200, about 1600 to about 2000, about 1600 to about 1800, about 1800 to about 3200, about 1800 to about 3000, about 1800 to about 2800, about 1800 to about 2600, about 1800 to about 2400, about 1800 to about 2200, about 1800 to about 2000, about 2000 to about 3200, about 2000 to about 3000, about 2000 to about 2800, about 2000 to about 2600, about 2000 to about 2400, about 2000 to about 2200, about 2200 to about 3200, about 2200 to about 3000, about 2200 to about 2800, about 2200 to about 2600, about 2200 to about 2400, about 2400 to about 3200, about 2400 to about 3000, about 2400 to about 2800, about 2400 to about 2600, about 2600 to about 3200, about 2600 to about 3000, about 2600 to about 2800, about 2800 to about 3200, about 2800 to about 3000, or about 3000 to about 3200 μg. In further embodiments, the selexipag iMTD and/or maintenance dose, on a twice daily basis, is about 200 to about 1600 μg. In other embodiments, the selexipag iMTD and/or maintenance dose is about 1000 μg to about 1600 μg twice daily. In yet further embodiments, the selexipag iMTD and/or maintenance dose, on a twice daily basis, is about 1400 to about 1600 μg.

Desirably, the selexipag starting dose is increased until the iMTD is reached. This period of increasing the dose may be determined by one skilled in the art. Typically, the time to reach the selexipag iMTD is at least about 1 week. In some embodiments, the time to reach the iMTD is at least about 2, about 3, about 4, about 5, about 6, about 7, about 8, about 9, about 10, about 11, about 12, about 13, about 14, about 15, or about 16 weeks. In other embodiments, the time to reach the iMTD is about 2 to about 16 weeks, about 2 to about 14 weeks, about 2 to about 12 weeks, about 2 to about 10, weeks, about 2 to about 8 weeks, about 2 to about 6 weeks, about 2 to about 4 weeks, about 4 to about 16 weeks, about 4 to about 14 weeks, about 4 to about 12 weeks, about 4 to about 10 weeks, about 4 to about 8 weeks, about 4 to about 6 weeks, about 5 to about 15 weeks, about 5 to about 10 weeks, about 6 to about 16 weeks, about 6 to about 14 weeks, about 6 to about 12 weeks, about 6 to about 10 weeks, about 6 to about 8 weeks, about 8 to about 16 weeks, about 8 to about 14 weeks, about 8 to about 12 weeks, about 8 to about 10 weeks, about 10 to about 16 weeks, about 12 to about 16 weeks, about 12 to about 14 weeks, or about 14 weeks to about 16 weeks. In further embodiments, the time to reach the selexipag iMTD dose is about 5 to about 10 weeks.

During the time to reach the iMTD, the selexipag dose is increased as determined by one skilled in the art until the iMTD is determined. In some embodiments, the selexipag dose is increased daily. In other embodiments, the selexipag dose is increased weekly. In further embodiments, the selexipag dose is increased monthly. Preferably, the selexipag dose is increased weekly (based on a 7-day week). When increased weekly, the selexipag dose may be administered on the same day each week or within 1 day within the scheduled dosing day. For example, if the selexipag dose in administered on a Monday, the next dose of selexipag may be administered on Sunday, Monday, or Tuesday of the following week. When increased monthly, the selexipag dose may be administered of the same day each month or within 3 days of the next scheduled dosing day. For example, if the selexipag dose in administered on January 7^{th} the next dose of selexipag may be administered on February 4^{th}, 5^{th}, 6^{th}, 7^{th}, 8^{th}, 9^{th}, or 10^{th}.

Once the iMTD is reached, it is maintained at a “maintenance dose” during a maintenance phase. The length of this maintenance phase may be determined by one skilled in the art and is, typically, at least about 14 weeks, and may continue as long as the patient is in need of therapy. In some embodiments, the maintenance phase is about 14, about 16, about 18, about 20, about 22, about 24, about 26, about 28, about 30, about 32, about 34, about 36, about 38, about 40, about 42, about 44, about 46, about 48, about 50, or about 52 weeks. In other embodiments, the maintenance phase is at least about 14 weeks. In further embodiments, the maintenance phase is at least about 26 weeks. In yet other embodiments, the maintenance phase is at least about 52 weeks.

Additional dose increases may occur following the maintenance phase as determined by those skilled in the art. However, any such increases desirably do not exceed 1600 μg twice daily.

The methods include administering a therapeutically effective amount of the selexipag. The term “therapeutically effective amount” as used herein, means that amount of active compound or pharmaceutical agent that elicits the biological or medicinal response in a human that is being sought by a researcher, medical doctor, or other clinician, which includes alleviation of the symptoms of the disease or disorder being treated. The selexipag may be administered once daily, twice daily, or thrice daily to achieve the therapeutically effective amount. In some aspects, the selexipag is administered once daily. In other aspects, the selexipag is administered twice daily. In further aspects, the selexipag is administered thrice daily. Preferably, the selexipag is administered twice daily to achieve the therapeutically effective amount.

As used herein, unless otherwise noted, the term “selexipag” refers to 2-{4-[(5,6-diphenylpyrazin-2-yl)(propan-2-yl)amino]butoxy}-N-(methanesulfonyl)acetamide of formula (II).

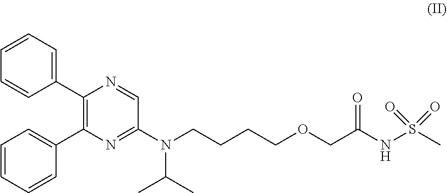 [SMILES: CC(C)N(CCCCOCC(=O)NS(C)(=O)=O)c1cnc(-c2ccccc2)c(-c2ccccc2)n1]

As used herein, “selexipag” also refers to amorphous or crystalline forms of selexipag, such as polymorphs thereof. In some embodiments, the selexipag is a crystalline form, such as a polymorph. In other embodiments, the selexipag is an amorphous form. In other embodiments, the selexipag is the Form I as described in U.S. Pat. Nos. 8,791,122 and 9,284,280, Form II as described in U.S. Pat. No. 9,340,516, or Form III as described in U.S. Pat. No. 9,440,931, all of which are incorporated by reference herein. The crystallinity may be determined by those skilled in the art using one or more techniques such as, e.g., single crystal x-ray diffraction, powder x-ray diffraction, differential scanning calorimetry, melting point, among others. “Selexipag” as used herein includes anhydrous or hydrates thereof. In certain embodiments, the selexipag is an anhydrous form. In other embodiments, the selexipag is a hydrate thereof. “Selexipag” as used herein further refers to solvates thereof. Such solvates include a molecule of a solvent bound through intermolecular forces or chemical bonds to one or more locations of the selexipag molecule.

The term “selexipag” may also include pharmaceutically acceptable salts thereof, which may readily be selected by those skilled in the art. A “pharmaceutically acceptable salt” is intended to mean a salt of selexipag that is non-toxic, biologically tolerable, or otherwise biologically suitable for administration to the subject. See, e.g., Berge, “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002, which are incorporated herein by reference. Selexipag can be used in the form of a free base or acid, but can also be used after forming into a pharmaceutically acceptable salt by a known method. When the selexipag is basic, examples of “salt” include salts of inorganic acids such as hydrochloric acid, sulfuric acid, nitric acid, phosphoric acid, hydrofluoric acid and hydrobromic acid, and salts of organic acids such as acetic acid, tartaric acid, lactic acid, citric acid, fumaric acid, maleic acid, succinic acid, methanesulfonic acid, ethanesulfonic acid, benzenesulfonic acid, p-toluenesulfonic acid, naphthalenesulfonic acid and camphorsulfonic acid. When the selexipag is acidic, examples of “salt” include alkali metal salts such as sodium salt and potassium salt, and alkali earth metal salts such as calcium salt. Geometrical isomers (Z form and E form) of selexipag or mixtures thereof are also contemplated. Selexipag is commercially available as understood to those skilled in the art. See, e.g., U.S. Pat. No. 7,205,302, which is incorporated by reference herein. For example, selexipag is available as Uptravi® and also is known as ACT-293987 or NS-304. Selexipag is an agonist of the prostacyclin receptor and may be prepared according to a process as disclosed in U.S. Pat. No. 7,205,302.

The present disclosure also contemplates the administration of selexipag metabolites. Desirably, the selexipag metabolite is metabolically active compound. Thus, in certain embodiments, the selexipag metabolite is of formula II-M1, 4-[(5,6-diphenylpyrazin-2-yl)(isopropyl)amino]butoxy}acetic acid. II-M1 is also known under the code name ACT-333679 or MRE-269. The preparation of selexipag is described in WO-2002/088084 (incorporated herein by reference). The preparation of polymorphic forms, i.e. the crystalline forms I, II, and III of the free base is disclosed in WO-2010/150865 (incorporated herein by reference); polymorphic forms of pharmaceutically acceptable salts are disclosed in WO-2011/024874 (incorporated herein by reference).

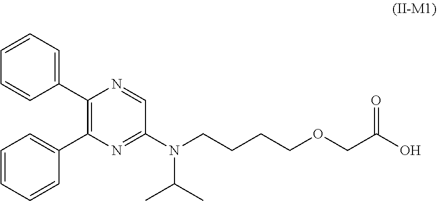 [SMILES: CC(C)N(CCCCOCC(=O)O)c1cnc(-c2ccccc2)c(-c2ccccc2)n1]

As used herein, unless otherwise noted, the terms “treating”, “treatment” and the like, shall include the management and care of a patient for the purpose of combating a disease, condition, or disorder. The terms “treating” and “treatment” also include the administration of the compounds or pharmaceutical compositions as described herein to (a) alleviate one or more symptoms or complications of the disease, condition or disorder; (b) prevent the onset of one or more symptoms or complications of the disease, condition or disorder; and/or (c) eliminate one or more symptoms or complications of the disease, condition, or disorder. Thus, “treating” and “treatment” include the administration of selexipag or pharmaceutical compositions containing the same to (a) alleviate one or more symptoms or complications of PAH; (b) prevent the onset of one or more symptoms or complications of PAH; and/or (c) eliminate one or more symptoms or complications of PAH.

One skilled in the art will recognize that, wherein the present disclosure is directed to methods of prevention, a patient in need thereof shall include any patient or patient who has experienced or exhibited at least one symptom of the disorder, disease or condition to be prevented. Further, a patient in need thereof may additionally be a patient who has not exhibited any symptoms of the disorder, disease or condition to be prevented, but who has been deemed by a physician, clinician or other medical profession to be at risk of developing said disorder, disease or condition. For example, the patient may be deemed at risk of developing a disorder, disease or condition (and therefore in need of prevention or preventive treatment) as a consequence of the patient's medical history, including, but not limited to, family history, pre-disposition, co-existing (comorbid) disorders or conditions, genetic testing, and the like.

The terms “subject” and “patient” are interchangeably used herein to refer to a human, who has been the object of treatment, observation or experiment. Preferably, the patient has experienced and/or exhibited at least one symptom of the disease or disorder to be treated and/or prevented. In some embodiments, the patient has PAH. In other embodiments, the patient is in World Health Organization functional class II or III prior to initiating the administration of selexipag. In further embodiments, the patient has a NT-proBNP>300 ng/L prior to the initiation of the administration of the selexipag. In yet other embodiments, the patient has not received treatment using an IP-receptor agonist, prostacyclin, or prostacyclin analog within at least 6 months prior to the initiation of the administration of selexipag.

MRI test images may be acquired at any time during selexipag treatment. For example, MRI test images may be acquired immediately after selexipag administration initiation, during the dose adjustment phase, or during the maintenance phase, or at the end of the study period. Desirably, the MRI test image(s) are acquired at least about 1 week after initiating treatment with selexipag. In some embodiments the MRI test image(s) are acquired at least about 4 weeks, about 8 weeks, about 12 weeks, about 15 weeks, about 18 weeks, about 21 weeks, about 24 weeks, about 26 weeks, about 30 weeks, about 50 weeks, about 52 weeks, or more after initiating treatment with selexipag. However, MRI test images may be acquired at any point during treatment with selexipag, such as during the lifetime of selexipag administration, in an effort to assess effectiveness of selexipag in treating the PAH or to evaluate the need to continue/discontinue/adjust treatment. On days when MRI analysis is obtained, the patient may be instructed to refrain from administering selexipag until after the MRI test images are obtained. During this treatment period, MRI test images may be taken on a regular basis such as yearly.

In some embodiments, the MRI images of the right ventricle, and the data obtained therefrom, may be used to adjust the selexipag treatment. In some embodiments, the MRI test images are compared with the baseline images to adjust the amount or frequency of selexipag administration. In other embodiments, the results of the MRI test images and their comparison with the baseline images can also aid in adjusting other parameters of the treatment, i.e., stopping selexipag treatment and/or switching or adding non-selexipag medications, among others.

The methods described herein also permit administering a background therapy at any stage, i.e., the prior to selexipag administration or during the initial dosing period, adjustment phase, or maintenance phase. The background therapy preferably includes pharmaceutical reagents that do not affect selexipag's activity on the patient. In some embodiments, the background therapy has been present for at least three months at a stable dose prior to administration of selexipag. In some embodiments, the background therapy includes administering one or more of a phosphodiesterase type 5 inhibitors, soluble guanylate cyclase stimulators, or endothelin receptor agonist to the patient. The amounts of the background therapy and need to continue/discontinue treatment may be determined and monitored by the attending physician.

### Administration Regimens

In the methods described herein, the amounts/doses of selexipag are safe, effective, or safe and effective. As used herein, unless otherwise noted, the term “safe” shall mean without undue adverse side effects (such as toxicity, irritation, or allergic response), commensurate with a reasonable benefit/risk ratio when used in the manner of this invention. Similarly, unless otherwise noted, the term “effective” means the efficacy of treatment has been demonstrated for the treatment of patients with pulmonary arterial hypertension when dosed in a therapeutically effective dose. In certain embodiments, the methods described herein are safe. In other embodiments, the methods described herein are effective. In further embodiments, the methods described herein are safe and effective. In yet other embodiments, the therapeutically effective amounts of selexipag are safe. In still further embodiments, the therapeutically effective amounts of selexipag are effective. In other embodiments, the therapeutically effective amounts of selexipag are safe and effective.

As used herein, unless otherwise noted, the term “clinically proven” (used independently or to modify the terms “safe” and/or “effective”) shall mean that proof has been proven by a Phase III or IV clinical trial that are sufficient to meet approval standards of U.S. Food and Drug Administration or similar study for market authorization by EMEA. Preferably, an adequately sized, randomized, double-blinded controlled study is used to clinically prove the effects of selexipag as compared to a placebo with the patient's condition assessed by techniques described herein.

As used herein, unless otherwise noted, the term “clinically proven effective” means the efficacy of treatment has been proven by a Phase III or IV clinical trial as statistically significant i.e., the results of the clinical trial are not likely to be due to chance with an alpha level less than 0.05 or the clinical efficacy results are sufficient to meet approval standards of U.S. Food and Drug Administration or similar study for market authorization by EMEA. For example, selexipag was clinically proven effective for the treatment of patients with pulmonary arterial hypertension in therapeutically effective doses as described herein, and as specifically set forth in the examples.

As used herein, unless otherwise noted, the term “clinically proven safe” means the safety of treatment has been proven by a Phase III or IV clinical trial by analysis of the trial data and results establishing that the treatment is without undue adverse side effects and commensurate with the statistically significant clinical benefit (e.g., efficacy) sufficient to meet approval standards of U.S. Food and Drug Administration or similar study for market authorization by Europe, the Middle East, and Africa (EMEA). For example, selexipag was clinically proven safe for the treatment of patients with pulmonary arterial hypertension when dosed in therapeutically effective doses as described herein, and as specifically set forth in the examples.

In certain aspects, methods of selling a drug product comprising selexipag are also provided. The terms “sale” or “selling” as used herein refers to transferring a drug product, e.g., a pharmaceutical composition or a dosage form, from a seller to a buyer. Thus, the methods include selling a drug product comprising selexipag, wherein the method comprises selling the drug product. In some embodiments, a drug product label for a reference listed drug for the drug product includes instructions for treating PAH. The methods also include offering for sale a drug product comprising selexipag. The term “offering for sale,” as used herein, refers to the proposal of a sale by a seller to a buyer for a drug product, e.g., a pharmaceutical composition or a dosage form. These methods comprise offering the drug product for sale.

In some embodiments, the present disclosure provides pharmaceutical drug products comprising clinically proven safe and clinically proven effective amount of selexipag, wherein the pharmaceutical product is packaged and wherein the package includes a label that identifies selexipag as a regulatory approved chemical entity and includes instructions for treating PAH and may also include MRI imaging data.

The term “drug product” refers to a product that contains an active pharmaceutical ingredient that has been approved for marketing by a governmental authority, e.g., the Food and Drug Administration or the similar authority in other countries. In some embodiments, the drug product comprises selexipag.

Similarly, “label” or “drug product label” refers to information provided to a patient which provides relevant information regarding the drug product. Such information includes, without limitation, one or more of the description of the drug, clinical pharmacology, indications (uses for the drug product), contraindication (who should not take the drug product), warnings, precautions, adverse events (side effects), drug abuse and dependence, dosage and administration, use in pregnancy, use in nursing mothers, use in children and older patients, how the drug is supplied, safety information for the patient, or any combination thereof. In certain embodiments, the label or drug product label provides instructions for treating PAH. In further embodiments, the label or drug product label identifies selexipag as a regulatory approved chemical entity and may also include MRI imaging data.

The term “reference listed drug” or “RLD” as used herein refers to a drug product to which new generic versions are compared to show that they are bioequivalent. It is also a medicinal product that has been granted marketing authorization by a member state of the European Union or by the Commission on the basis of a completed dossier, i.e., with the submission of quality, pre-clinical and clinical data in accordance with Articles 8(3), 10a, 10b or 10c of Directive 2001/83/EC and to which the application for marketing authorization for a generic/hybrid medicinal product refers, by demonstration of bioequivalence, usually through the submission of the appropriate bioavailability studies.

In certain embodiments, the drug product is an ANDA drug product, a supplemental New Drug Application drug product, or a 505(b)(2) drug product. In the United States, a company seeking approval to market a generic equivalent must refer to the RLD in its Abbreviated New Drug Application (ANDA). For example, an ANDA applicant relies on the FDA's finding that a previously approved drug product, i.e., the RLD, is safe and effective, and must demonstrate, among other things, that the proposed generic drug product is the same as the RLD in certain ways. Specifically, with limited exceptions, a drug product for which an ANDA is submitted must have, among other things, the same active ingredient(s), conditions of use, route of administration, dosage form, strength, and (with certain permissible differences) labeling as the RLD. The RLD is the listed drug to which the ANDA applicant must show its proposed ANDA drug product is the same with respect to active ingredient(s), dosage form, route of administration, strength, labeling and conditions of use, among other characteristics. In the electronic Orange Book, there is a column for RLDs and a column for reference standards. In the printed version of the Orange Book, the RLDs and reference standards are identified by specific symbol.

In Europe, Applicants identify in the application form for its generic/hybrid medicinal product, which is the same as an ANDA or supplemental NDA (sNDA) drug product, the reference medicinal product (product name, strength, pharmaceutical form, marketing authorization holder (MAH, first authorization, Member State/Community), which is synonymous with a RLD, as follows:

1. The medicinal product that is or has been authorized in the European Economic Area (EEA), used as the basis for demonstrating that the data protection period defined in the European pharmaceutical legislation has expired. This reference medicinal product, identified for the purpose of calculating expiry of the period of data protection, may be for a different strength, pharmaceutical form, administration route or presentation than the generic/hybrid medicinal product.

2. The medicinal product, the dossier of which is cross-referred to in the generic/hybrid application (product name, strength, pharmaceutical form, MAH, marketing authorization number). This reference medicinal product may have been authorized through separate procedures and under a different name than the reference medicinal product identified for the purpose of calculating expiry of the period of data protection. The product information of this reference medicinal product will, in principle, serve as the basis for the product information claimed for the generic/hybrid medicinal product.

3. The medicinal product (product name, strength, pharmaceutical form, MAH, Member State of source) used for the bioequivalence study(ies) (where applicable).

The different abbreviated approval pathways for drug products under the Food, Drug, and Cosmetics (FD&C) Act are the abbreviated approval pathways described in sections 505(j) and 505(b)(2) of the FD&C Act (21 U.S.C. 355(j) and 21 U.S.C. 355(b)(2), respectively).

According to the FDA (“Determining Whether to Submit an ANDA or a 505(b)(2) Application Guidance for Industry,” U.S. Department of Health and Human Services, October 2017, pp. 1-14, the contents of which is incorporated herein by reference), NDAs and ANDAs can be divided into the following four categories:

(1) A “stand-alone NDA” is an application submitted under section 505(b)(1) and approved under section 505(c) of the FD&C Act that contains full reports of investigations of safety and effectiveness that were conducted by or for the applicant or for which the applicant has a right of reference or use.

(2) A section 505(b)(2) application is an NDA submitted under section 505(b)(1) and approved under section 505(c) of the FD&C Act that contains full reports of investigations of safety and effectiveness, where at least some of the information required for approval comes from studies not conducted by or for the applicant and for which the applicant has not obtained a right of reference or use.

(3) An ANDA is an application for a duplicate of a previously approved drug product that was submitted and approved under section 505(j) of the FD&C Act. An ANDA relies on the FDA's finding that the previously approved drug product, i.e., the reference listed drug (RLD), is safe and effective. An ANDA generally must contain information to show that the proposed generic product (a) is the same as the RLD with respect to the active ingredient(s), conditions of use, route of administration, dosage form, strength, and labeling (with certain permissible differences) and (b) is bioequivalent to the RLD. An ANDA may not be submitted if studies are necessary to establish the safety and effectiveness of the proposed product.

(4) A petitioned ANDA is a type of ANDA for a drug product that differs from the RLD in its dosage form, route of administration, strength, or active ingredient (in a product with more than one active ingredient) and for which FDA has determined, in response to a petition submitted under section 505(j)(2)(C) of the FD&C Act (suitability petition), that studies are not necessary to establish the safety and effectiveness of the proposed drug product.

A scientific premise underlying the Hatch-Waxman Act is that a drug product approved in an ANDA under section 505(j) of the FD&C Act is presumed to be therapeutically equivalent to its RLD. Products classified as therapeutically equivalent can be substituted with the full expectation that the substituted product will produce the same clinical effect and safety profile as the prescribed product when administered to patients under the conditions specified in the labeling. In contrast to an ANDA, a section 505(b)(2) application allows greater flexibility as to the characteristics of the proposed product. A section 505(b)(2) application will not necessarily be rated therapeutically equivalent to the listed drug it references upon approval

The methods may also comprise, consist of, or consist essentially of placing selexipag into the stream of commerce. In certain embodiments, selexipag includes a package insert that contains instructions for treating PAH and may also include MRI imaging data.

In further aspects, described herein are methods of selling pharmaceutical compositions containing selexipag comprising, consisting of, or consisting essentially of placing the pharmaceutical composition into the stream of commerce. In certain embodiments, the pharmaceutical composition includes a package insert that contains instructions for treating PAH and may also include MRI imaging data.

In still further aspects, described herein are methods of offering for sale selexipag comprising, consisting of, or consisting essentially of offering to place selexipag into the stream of commerce. In certain embodiments, selexipag includes a package insert that contains instructions for treating PAH and may also include MRI imaging data.

### Formulations/Compositions

Pharmaceutical compositions containing selexipag can be prepared by intimately mixing the compound or compounds with a pharmaceutical carrier according to conventional pharmaceutical compounding techniques. As used herein, the terms “composition” and “formulation” are used interchangeably and encompass a product comprising the specified ingredients in the specified amounts, as well as any product, such as a pharmaceutical product, which results, directly or indirectly, from combinations of the specified ingredients in the specified amounts. A summary of pharmaceutical compositions can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

Such compositions may be administered to a patient neat or in a mixture with a pharmaceutically acceptable non-toxic inert carrier, for example, as a pharmaceutical composition containing the compound at a level of 0.1% to 99.5 wt %, preferably 0.5% to 90%, based on the total weight of the composition. As a carrier, one or more of auxiliary agents for formulations such as solid, semi-solid and liquid diluent, filler and other auxiliary agents for drug formulations may be used. It is desirable that a pharmaceutical composition is administered as a unit dosage form.

Selexipag may be administered by a number of routes as determined by those skilled in the art. Preferably, selexipag is administered by route that is suitable for selexipag. In some embodiments, selexipag is administered orally, parenterally, or by inhalation, or any combination thereof. In other embodiments, selexipag is administered orally. In further embodiments, selexipag is administered by inhalation. In other embodiments, selexipag is administered parenterally. In further embodiments, selexipag is administered orally in the form of one or more tablets.

In some embodiments, selexipag is administered as injections or infusions such as subcutaneous or intravenous injections. For intravenous administration, the selexipag pharmaceutical product is a sterile solution. Injectable suspensions or solutions may be prepared utilizing aqueous carriers along with appropriate additives. For intravenous administration, the carrier will usually consist of sterile water and other ingredients which increase solubility or preservation. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin. Isotonic preparations which may contain suitable preservatives are employed when intravenous administration is desired. In some embodiments, the carrier used in intravenous formulations comprises sterile water.

For oral administration, each preparation may be solid or liquid. Preferably, the oral forms of selexipag as described herein are solids. Examples of solid formulations include, for example, pastilles, thin films, pastes, lozenges, granules, powders, capsules, pills such as caplets, gelcaps, tablets, and capsules (each including immediate release, timed release and sustained release pills). Preferably, the oral compositions are administered as tablets, i.e., desirably, the pharmaceutical product comprises a tablet. If desired, tablets or caplets may be sugar coated or enteric coated by standard techniques or otherwise compounded to provide a dosage form affording the advantage of prolonged action. For preparing solid compositions such as tablets, selexipag is mixed with a pharmaceutical carrier/additive such as starches, sweeteners such as sugars, diluents, coloring agents, granulating agents, preservatives, lubricants, flavoring agents, binders, disintegrating agents and the like. For tablets, conventional tableting ingredients such as corn starch, lactose, sucrose, sorbitol, talc, stearic acid, magnesium stearate, dicalcium phosphate or gums, and other pharmaceutical diluents, e.g., water, ethanol, glycerol, or the like, may be used. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrating agents include, without limitation, starch, methyl cellulose, agar, bentonite, xanthan gum and the like. The liquid forms in which the compositions of the present disclosure may be incorporated for administration by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. The liquid oral preparations independently contain selexipag and one or more of suitable carriers/additives such as water, glycols, oils, alcohols, flavoring agents, preservatives, stabilizers, coloring agents and the like.

To prepare pharmaceutical compositions of the present disclosure, selexipag may be intimately admixed with a pharmaceutical carrier according to conventional pharmaceutical compounding techniques, which carrier may take a wide variety of forms depending of the form of preparation desired for administration (e.g., oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain, the disclosure of which is hereby incorporated by reference.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc., the disclosures of which are hereby incorporated by reference.

### Aspects

Aspect 1: A method for treating pulmonary arterial hypertension in a patient in need thereof, comprising:
(a) performing magnetic resonance imaging (MRI) on the right ventricle of the patient to provide a MRI baseline image;
(b) administering a therapeutically effective amount of selexipag;
(c) performing MRI on the right ventricle of the patient to provide a MRI test image; and
(d) comparing the MRI baseline image with the MRI test image.

Aspect 2: The method of Aspect 1, wherein step (d) is performed at least about 26 weeks after initiating the administration of the selexipag.

Aspect 3: The method of Aspect 1 or 2 wherein the treatment is adjusted based on step (d).

Aspect 4: The method of any one of Aspects 1 to 3, wherein, prior to initiating the administration of the selexipag, the patient is in World Health Organization functional class II or III or has a NT-proBNP>300 ng/L prior to the initiation of the administration of the selexipag.

Aspect 5: The method of any one of the preceding Aspects, wherein the patient has not received treatment using an IP-receptor agonist, prostacyclin, or prostacyclin analog within at least 6 months prior to the initiation of the administration of the selexipag.

Aspect 6: The method of any one of the preceding Aspects, wherein the selexipag is administered at a starting dose and is increased to determine an individual maximum tolerated dose (iMTD).

Aspect 7: The method of Aspect 6, wherein the starting dose is about 200 μg twice daily.

Aspect 8: The method of Aspect 6 or 7, wherein the iMTD is from about 200 μg to about 1600 μg twice daily.

Aspect 9: The method of any one of Aspects 6 to 8, wherein the iMTD does not exceed about 1600 μg twice daily.

Aspect 10: The method of any one of Aspects 6 to 9, wherein the iMTD is maintained during a maintenance phase following a dose adjustment phase.

Aspect 11: The method of any one of the preceding Aspects, wherein the patient receives background therapy comprising phosphodiesterase type 5 inhibitors, soluble guanylate cyclase stimulators, or endothelin receptor agonist, wherein such therapy is present for at least three months at a stable dose prior to administration of selexipag.

Aspect 12: The method of any one of the preceding Aspects, wherein the pulmonary arterial hypertension is idiopathic pulmonary arterial hypertension, heritable pulmonary arterial hypertension, pulmonary arterial hypertension associated with connective tissue disease, or pulmonary arterial hypertension associated with congenital heart disease, with simple systemic-to-pulmonary shunt at least 1 year after surgical repair.

Aspect 13: The method of any one of the preceding Aspects, wherein the patient's right ventricular stroke volume (RVSV), as measured by MRI, increases after about 26 weeks following the selexipag initiation.

Aspect 14: The method of Aspect 13, wherein the increase is from about 8 mL to about 12 mL.

Aspect 15: The method of any one of the preceding Aspects, wherein the patient's right ventricular ejection fraction (RVEF), as measured by MRI, increases after about 26 weeks following the selexipag initiation.

Aspect 16: The method of any one of the preceding Aspects, wherein the patient's right ventricular global longitudinal strain (RVGLS), as measured by MRI, improves after about 26 weeks following the selexipag initiation.

Aspect 17: The method of any one of the preceding Aspects, wherein the selexipag is inhaled, orally administered, or parenterally administered.

Aspect 18: The method of Aspect 17, wherein the selexipag is inhaled.

Aspect 19: The method of Aspect 17, wherein the selexipag is parenterally administered.

Aspect 20: The method of Aspect 19, wherein the parenteral administration is subcutaneous or intravenous.

Aspect 21: The method of Aspect 17, wherein the selexipag is an orally administered.

Aspect 22: The method of Aspect 21, wherein the selexipag is the form of a tablet.

Abbreviations

| | |
|---|---|
| 6MWD | 6-minute walking distance |
| 6MWT | 6-minute walking test |
| AE | Adverse event |
| AESI | Adverse event of special interest |
| ALT | Alanine aminotransferase |
| ANCOVA | Analysis of covariance |
| AST | Aspartate aminotransferase |
| AV | Atrioventricular |
| BDI | Borg dyspnea index |
| CI | Confidence interval |
| CV | Coefficient of variation |
| 4D | 4-dimensional |
| ECG | Electrocardiogram |
| Echo | Echocardiogram |
| EOS | End of Study |
| EOT | End of Treatment |
| EQ-5D | EuroQol 5-Dimension scale |
| ERA | Endothelin receptor antagonist |
| FAS | Full Analysis Set |
| FC | Functional class |
| FIESTA | Fast Imaging Employing Steady state |
| | Acquisition |
| FISP | Fast Imaging with Steady-state Precession |
| HCG | Human chorionic gonadotropin |
| HIV | Human immunodeficiency virus |
| IAP | Image Acquisition Protocol |
| ICH | International Council for Harmonisation of |
| | Technical Requirements |
| IMD | Individual maintenance dose |
| INR | International normalized ratio |
| IP | Prostacyclin |
| IT-OT | Inflow tract - outflow tract |
| LPC | Local Phase Correction |
| LV | Left ventricle, left ventricular |
| LVEDV | Left ventricular (LV) end diastolic volume |
| LVEF | Left ventricular (LV) ejection fraction |
| LVESV | Left ventricular (LV) end systolic volume |
| MCV | Mean corpuscular volume |
| MPA | Main pulmonary artery |
| mPAP | Mean pulmonary arterial pressure |
| MR | Magnetic resonance |
| MRI | Magnetic resonance imaging |
| NT-proBNP | N-terminal-pro-hormone brain natriuretic |
| | peptide |
| PA | Pulmonary artery |
| PAH | Pulmonary arterial hypertension |
| PAWP | Pulmonary artery wedge pressure |
| PPS | Per-Protocol Set |
| PT | Prothrombin time |
| PVR | Pulmonary vascular resistance |
| RA | Right atrium, right atrial |
| RBC | Red blood cell |
| RF | Radiofrequency |
| RHC | Right heart catheterization |
| RR | time R-top to R-top time interval |
| RV | Right ventricle, right ventricular |
| RVEDV | Right ventricular (RV) end diastolic volume |
| RVEF | Right ventricular (RV) ejection fraction |
| RVESV | Right ventricular (RV) end systolic volume |
| RVGLS | Right ventricular (RV) global longitudinal |
| | strain |
| RVSV | Right ventricular (RV) stroke volume |
| SAE | Serious adverse event |
| SBP | Systolic blood pressure |
| SD | Standard deviation |
| SS | Safety Set |
| SSFP | Steady State Free Precession |
| SUSAR | Suspected unexpected serious adverse |
| | reaction |
| T3 | tri-iodothyronine |
| T4 | thyroxine |
| TSH | thyroid-stimulating hormone |
| VLA | Vertical long axis |
| WBC | White blood cell |
| WHO | World Health Organization |
| WU | Wood unit |

### EXAMPLES

### Example 1

This protocol defines how cardiac MRI images should be acquired.

A. MRI Conditional Pacemakers

For patients with an MRI-conditional pacemaker, it is indispensable to ensure it is indeed suitable for cardiac MRI as described in this imaging protocol, taking into account available scanner characteristics (e.g., field strength) and strictly follow manufacturer instructions.

B. Equipment
1.5 or 3 Tesla MRI system capable of cardiovascular imaging.
Cardiac receiver coils, or other phased-array receiver coils that can be used for cardiac imaging, such as a phased-array body coil.
MR compatible leads for ECG or Vector Cardiogram gating.
   Scanner software: Licenses for cardiac and flow MR pulse sequences.
   If contrast medium is to be used, it should be used after the cine cardiac MRI sequences are complete.

C. Patient Preparation

Patient should avoid eating 2 hours prior to the exam, in order to decrease motion artifacts due to intestinal motion. Patient should lie in supine position. Ensure proper ECG lead placement with good ECG/VectorCardiographic signal.

The R-wave should be correctly recognized by the scanner software, also when the patient is moved to the magnet center. Verify correct R-wave triggering also during scanning. Patient should be familiar with breathhold instructions: the best reproducible breathhold is at relaxed expiration. Record the heart rate during the cine imaging, and also during the flow imaging.

D. MRI Pulse Sequences

(i) Localizing the Heart, Pulmonary Artery and Ascending Aorta

SSFP imaging is recommended. Typical settings are a repetition time of 3.2 ms, echo time of 1.6 ms, flip angle in a range of 40 to 60 deg, slice thickness of 6 mm, and acquisition matrix of 256×96. All planning images must be acquired in the end-diastolic phase of the cardiac cycle, while the patient holds his breath at relaxed expiration. For this localizing and planning, other pulse sequences may be used if acquisition is in end-diastole.

(ii) Cine Imaging in Breathhold by SSFP (FIESTA, Balanced Fast Field Echo, or True-FISP)

Breathhold duration may be shortened by using parallel imaging, which is optional. The following parameters are applied for the 4-chamber view, short-axis stack, LV 3-chamber view, RV Inflow tract-outflow tract view, and axial stack of RV: slice thickness of 6 mm, slice gap in short-axis stack ranging between 0 and 4 mm, with a 4 mm maximal, a field of view of 280×320 mm (may be adapted to patient size), matrix of at least 256×128, flip angle in a range of 40 to 70 deg, #phase-encoding lines/beat of 11 to 15 (also called “#views per segment”), #temporal phases of at least 25 (temporal resolution <5% of RR time), and ECG gating that is retrospectively.

(iii) MR Velocity Quantification

Phase-contrast velocity imaging is performed during continued normal breathing. The pulse sequence is an ECG-gated, spoiled gradient-echo sequence, with through-plane velocity encoding and a velocity sensitivity of 120 cm/s for the main pulmonary artery (150 cm/s for the aorta). The orientation of the image plane must be orthogonal to the main pulmonary artery (or ascending aorta).

The flow sequence is run with the following parameters (field of view adaptable to patient): velocity sensitivity of 120 cm/s for the main pulmonary artery, velocity sensitivity of 150 cm/s for the aorta, slice thickness of 6 to 8 mm, field of view of 240×320 mm², matrix size of 140×256, echo time of 4.8 ms, repetition time of 11 ms (maximum), temporal resolution of 22 ms (maximum), and flip angle of 25 deg.

E. 4-Chamber Cine and the Stack of Short-Axis Cines

A: coronal; B: transverse; C. vertical long-axis; D. basal short-axis; E: VLA; F. 4-chamber; G: VLA; H: 4-chamber; I: mid-short-axis

Consecutive steps in the planning of the stack of short-axis cines:

1. The line on the coronal view (“A”) through the ventricles defines the transverse view (“B”).

2. The line on the transverse view, running parallel to the septum and through the LV apex, defines the VLA view (“C”).

3. The line on the VLA view defines the basal short-axis view (“D”).

4. On D, the 1^{st} planning line for the 4 chamber view is drawn between the LV center and the RV right angle.

5. On E, the 2^{nd} planning line connects the LV apex with the middle of the mitral valves.

6. Planning lines both on D and on E are used simultaneously in order to acquire the 4-chamber cine (“F”).

7. A stack of parallel planning lines are applied both on the VLA (“G”) and the 4-chamber (“H”), parallel to the AV groove and orthogonal to the septum. The most basal short-axis slice must be located immediately on the myocardial side of the atrio-ventricular junction. This delivers the orientation of the stack of short-axis cine images fully covering the ventricles (mid-slice in I).

8. The stack of short-axis cines must cover the right and left ventricle completely, from the apex till the most basal part.

F. Flow Measurement: Technical Requirements

At follow-up, the same MRI scanner and the same pulse sequences should be used as were used at the baseline measurement. Posterior and anterior RF receiver coils should be enabled in the image Field Of View. ECG triggering must be retrospectively. Verify correct triggering during the flow measurement. The imaging plane must be at the isocenter of the magnet. The imaging plane must be at the correct anatomical position: the cross section of main pulmonary artery (or aorta) must be clearly delineated in the magnitude and velocity images throughout the cardiac cycle. No infolding (“wrap-around” or “fold-over”) artifacts in the images are allowed. If there is aliasing in the velocity map, repeat the flow measurement with higher encoding velocity.

It is recommended to plot the flow curve immediately after the acquisition, while the patient is still in the magnet. If necessary, repeat the flow measurement.

Vendor-Specific Requirements

Philips
LPC filter must be set on OFF (choose NO on the tab ‘postproc’ of the Philips examcard).
Default gradient mode.

Siemens
Gradient mode set to ‘normal’.
Area and Skyra: Velocity compensation of phase-encoding direction set ‘off’.

GE
Flow optimization must be ON.
Use the CINE phase-contrast sequence with the Control Variable ‘magnitude weighting’ disabled.

G. Flow Quantification in the Main Pulmonary Artery

A: transverse image; B: oblique-sagittal image; C: extra localizer with planning line for flow; D: oblique-sagittal image with planning line for flow; E: pulmonary artery magnitude image; F: pulmonary artery velocity image

Localizing the Main Pulmonary Artery:

1. Start with transverse view (A) showing the main and the right pulmonary artery.

2. An image is prescribed perpendicular to A that includes the main pulmonary artery. This results in an oblique-sagittal image as shown in B.

3. Perpendicular to B, and through the pulmonary artery as shown by the planning line on B, an extra localizer image (C) is acquired.

4. Use the planning lines on C and on D simultaneously, to obtain the imaging plane for flow measurement (E).

5. On plane E, the through-plane flow in the main pulmonary artery is measured.

6. Panel F shows the velocity image in mid-systole.

The stroke volume, as calculated form the pulmonary artery flow, is a primary endpoint of the study.
Verify correct positioning of the imaging plane for flow and determine if the pulmonary artery cross section clearly delineated through the cardiac cycle.
Verify correct triggering by inspecting the velocity images in cine mode.
Infolding artifacts must be avoided on the images of the flow measurement.
No aliasing allowed in the velocity images.
Repeat the flow measurement if necessary.

H. Flow Quantification in the Aorta

A: coronal; B: transverse; C: magnitude image; D: velocity image

1. For the aortic flow measurement, the location is a pure transverse plane where the ascending aorta is in the feet-head direction (see panel A), and where the right pulmonary artery is inplane.

2. This results in the imaging plane (B), on which flow is measured.

3. The flow measurement contains magnitude and velocity images, as shown in panels C and D in midsystole.

4. Correct triggering is verified by inspecting the velocity images in cine mode. Infolding artifacts must be avoided on the images of the flow measurement. No aliasing allowed in the velocity images. Repeat the flow measurement if necessary.

I. Long axis 3-chamber cine, and the RV IT-OT cine

A: basal short-axis; B: 4-chamber; C: LV 3-chamber: D: basal short-axis: E: 4-chamber; F: RV IT-OT

1. The LV 3-chamber cine (C) is planned on the most basal short-axis slice

(A).

2. The planning line is oriented through the center of the LV and the middle of the aortic root.

3. On the 4-chamber cine (B) the slice should pass through the apex of the LV.

4. The RV IT-OT cine (F) is planned on the most basal short-axis slice (D).

5. The planning line is oriented through the center of the RV tricuspid valves, and the pulmonary valves.

6. On the 4-chamber cine (E) the slice should pass through the apex of the RV.

J. Stack of axial cines, covering the RV volumes

A: coronal; B: axial (=transverse)

1. Acquire a stack of breathhold cine series in axial orientation. This stack must cover the full RV volume completely, from the diaphragm till the pulmonary artery.

2. The planning line on the coronal image (A) defines the image plane for one of the axial cine series (B).

3. The planning images should be acquired at relaxed expiration, in the end-diastolic phase of the cardiac cycle.

4. For the axial cines, use the same sequence and the same temporal resolution as were used for the short-axis cines.

### Example 2

A. Study Design

(i) Overall Design

This is an open-label, multicenter, single-arm, interventional study to assess the effect of selexipag in adult subjects (≥18 to <65 years) with a diagnosis of PAH up to 26 and 52 weeks on treatment. Patients will have PAH (idiopathic PAH, heritable PAH, PAH caused by drugs or toxins, PAH associated with connective tissue disease or congenital heart disease) and no contraindication to magnetic resonance imaging (MRI) or to selexipag.

The duration of individual participation will be approximately 60 weeks. The study will be conducted in 3 phases: a 28-day screening phase, a 52-week intervention phase (which will include an initial 12-week up-titration period), and a post-intervention safety follow-up period of at least 30 days. Primary and secondary objectives will be evaluated up to Week 26 and exploratory objectives will be evaluated up to Week 52. Following are detailed descriptions of the study periods:
Screening period: Informed consent signature marks both the start of the study and the start of the screening period. This period serves to assess eligibility (medical history, treatments, blood tests) and to perform baseline efficacy assessments (MRI, Echo, 6MWD, WHO FC, NT-proBNP, and exploratory blood tests). Screening may last up to 28 days. A subject who is temporarily ineligible may be re-screened. In case MRI, Echo, 6MWT, or WHO FC assessment was made as part of routine practice in a way that fully complies with the study requirements, and within 28 days before Day 1, these data can be used for the study.
Treatment period: Starts with the first dose of study drug (Day 1 of study) and ends with EOT on the day of the last dose of study drug which is at Week 52±7 days or at premature discontinuation of study drug.
Day 1: Day 1 is defined as the day when a patient receives the first dose of study drug. First dosing should occur at the site. Day 1 may occur once screening assessments are completed, and no later than 28 days after informed consent signature. For re-screened subjects, all screening assessments must have been made (or repeated) within 28 days before Day 1.
Up-titration: During this period, study drug will be up-titrated from Day 1 to the end of Week 12 (Day 84). Weekly phone calls from the site to patient will be performed to guide the up-titration and collect safety information. At the end of Week 4 (+7 days) and Week 12 (+7 days) and patients will return to the site for a return/dispensing visit; no assessments will be performed at these site visits.
Maintenance: During this period, subjects will receive study drug at their individual maintenance dose (IMD) from the start at Week 13 to EOT (scheduled at the end of Week 52, ±7 days). Monthly phone calls (except when site visits take place) from the site to the patient will be made to monitor patient's safety by collecting information on concomitant medications, AEs and SAEs. At Week 26 (Day 168 to 196), a site visit is scheduled to assess MRI, Echo, 6MWT, WHO FC, blood draw for secondary and exploratory endpoints. At Week 39 (+14 days), patients will return to the site for a study drug return/dispensing visit; no assessments will be performed at this site visits.
EOT: EOT should occur at end of Week 52±2 weeks (Day 350 to 378). At this visit, postbaseline assessments will be performed (MRI, 6MWT, WHO FC, blood draw). In case of premature EOT before Week 16, postbaseline efficacy assessments will be optional and safety assessments will be mandatory. In case of premature EOT after Week 16 but before Week 26, all postbaseline assessments will be performed.
Safety follow-up: The safety follow-up will start the day after the last study drug dose and end with the safety follow-up telephone call (EOS visit) at least 30 days after the last dose.
Unscheduled visits: Unscheduled visits will be allowed; however, any AE/SAE reported during an unscheduled visit should still be recorded.

Eligible subjects will be treated with selexipag for 52 weeks. Dosing with selexipag will start at 200 μg twice daily. On Day 1, the subject will receive only 1 dose, and at each dose change, the first intake of the new dose should be taken in the evening. The site will call the subject once a week from the end of Week 1 to the end of Week 12 and decide whether to increase the dose by 200 μg twice daily if possible. Up-titration will be flexible and can be adapted in case of adverse effects that cannot be relieved with symptomatic treatment. In this case, the site may either postpone up-titration by 1 week or down-titrate study drug. The dose reached at end of Week 12 will be considered the subject's IMD and will be maintained until end of treatment.

Efficacy assessments will include MRI, Echo, 6MWD, WHO FC, NT-proBNP, and risk stratification. Safety assessments will include the monitoring of AEs, clinical laboratory evaluations, and pregnancy testing in female subjects of childbearing potential. Exploratory assessment will include biomarkers and the subjects' experience evaluated using a questionnaire.

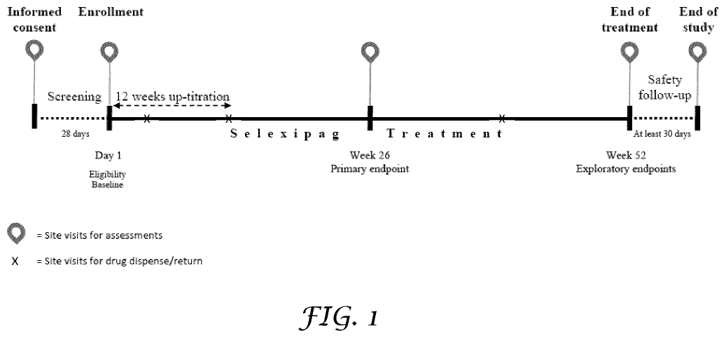

A diagram of the study design is provided in FIG. 1. This study will contain a 12-week up-titration period. The goal of the up-titration is to ensure that each subject reaches his/her individual highest tolerated dose; i.e., without unmanageable prostacyclin-associated adverse effects.

Changes in circulating biomarkers from baseline to Week 26 will be explored. Additional exploratory biomarkers may be measured after the EOS and will be based on the latest scientific evidence regarding RV function and structure at the time of laboratory analysis. No genetic testing of any kind will be performed.

(ii) End of Study Definition

EOS Definition: The EOS is defined as the day of the last study assessment shown in the schedule of activities (Table 1) for the last subject in the study.

| | | SELEXIPAG TREATMENT | | | | | | | |
|---|---|---|---|---|---|---|---|---|---|
| | | Up-titration to IMD | | | | | | | |
| | | (Day 1 to Week 12)^{a} | | | Maintenance with IMD | | | | |
| | | | | Study | (Weeks 13 to 52) | | | | FOLLOW- |
| | | | | drug | | | Study | | UP |
| | | | Phone | dispense/ | Phone | Primary | drug | | EOS |
| | SCREENING | Treatment | calls | return | calls #13 | efficacy | dispense/ | | (Phone |
| | Screening | initiation | #1 to #12 | #1 and #2 | to #19 | assessments^{p} | return #3 | EOT | call #20) |
| | Time phase | | | | | | | | |
| | | | | Week 4 | | | | Week 52 ± | |
| PERIODS | | | | (+7 days) | | | | 7 days | |
| Visit name | | | | and | | | | or at | At least 30 |
| Study | Day −28 to | | Weekly | Week 12 | Monthly^{q} | Week 26 | Week 39 | premature | days after |
| procedure | Day 1 | Day 1 | (± 3 days) | (+7 days) | (± 5 days) | (±14 days^{b,r}) | (±14 days) | EOT^{b} | EOT |

Sereening/Administ rative

| | | | | | | | |
|---|---|---|---|---|---|---|---|
| Informed | X | | | | | | |
| consent ^{c} | | | | | | | |
| Inclusion/ | X | | | | | | |
| exclusion | | | | | | | |
| criteria ^{d,o} | | | | | | | |
| Demographics, | X | | | | | | |
| PAH | | | | | | | |
| characteristics | | | | | | | |
| Medical history | X | | | | | | |
| Concomitant | X | X | X | X | X | X | X |
| medication | | | | | | | |
| Contraceptive | X | X | X | X | X | X | X |
| methods^{e} | | | | | | | |
| Pregnancy test^{e} | X ^{f} | X ^{g} | Monthly^{h} | X | X ^{f} | X ^{f} | X ^{h,i} |

Study Drug Administration

| | | | | | |
|---|---|---|---|---|---|
| Dispense/ | X | X | X | X | |
| administer | | | | | |
| study drug | | | | | |
| Return study | | X | X | X | X |
| drug | | | | | |
| Study drug | | X | X | X | X |
| accountability | | | | | |

Efficacy Evaluations

| | | | |
|---|---|---|---|
| MRI | X ^{j} | X ^{k} | X |
| Echo | X ^{j} | X ^{k} | |
| 6MWT | X ^{j} | X ^{k} | X |
| including Borg | | | |
| CR 10 ® | | | |
| WHO FC | X ^{j} | X ^{k} | X |
| Risk | X | X ^{k} | |
| stratification | | | |

Safety Evaluations

| | | | | | | | |
|---|---|---|---|---|---|---|---|
| Vital signs (BP, | X | X | | | X | X | |
| HR, weight) | | | | | | | |
| Patient's | | X | | | X | X | |
| experience^{l} | | | | | | | |
| Physical | X | | | | X^{l} | X | |
| examination | | | | | | | |
| Blood drawⁿ | X | | | | X | X | |
| AEs and SAEs | X | X | X | X | X | X | X |

^{a}In principle, the maintenance dose can be reached after 8 weeks. Additional time is given to allow slower up-titration.
^{b}Patients must not take the morning study drug dose before any visit-related procedure.
^{c} Must be signed before first study-related activity.
^{d} For eligibility criteria based on blood laboratory results, the decision to enroll a patient can be made on the basis of local laboratory results obtained during screening; however, the blood samples must still be sent to the central laboratory for analysis.
^{e}For females of childbearing potential.
^{f} Serum pregnancy test.
^{g} Urine pregnancy test at site.
^{h} Urine pregnancy test at patient's home. The site will call female patients of childbearing potential monthly to check the pregnancy test result. A window of ±5 days for each pregnancy test/phone call will be allowed.
ⁱ Test to be done within 30 to 37 days after EOT.
^{j} If MRI, Echo, 6MWT, or WHO FC assessments were done as part of routine practice in a way that fully complies with the study requirements, and within 28 days before Day 1, these data can be used for the study.
^{k} Optional in case of premature EOT before end of Week 16 (Day 112).
^{l}Only if EOT.
^{m}Patient's experience refers to the EQ-5D-3L questionnaire.
ⁿFor safety laboratory analyses, NT-proBNP and biomarkers.
^{o} If the results for the screening blood samples from the central laboratory are not available in time for Day 1 visit, an additional blood sample may be drawn to verify eligibility based on a local laboratory test.
^{p}Including premature EOT. In case of premature EOT, the EOT visit should take place within 7 days after the decision to end study drug.
^{q}Except for month of Week 26 and Week 52.
^{r}In case of epidemic situation, e.g., COVID-19, the Week 26 visit may be delayed up to an additional 28 days (Week 26 +6/−2 weeks)

Study Completion Definition for an Individual Subject: The EOS for an individual subject is the end of the safety follow-up, which is planned at least 30 days after last study drug intake (also applies to subjects who prematurely discontinue study drug). For subjects lost to follow-up, the EOS is the last contact with the site. In case of death, death is the EOS.

B. Study Population

Screening for eligible subjects will be performed within 28 days before administration of the study drug. If a subject's clinical status changes (including any available laboratory results or receipt of additional medical records) after screening but before the first dose of study drug is given such that he/she no longer meets all eligibility criteria, then the subject should be excluded from participation in the study.

Eligibility assessment may be made on the basis of local laboratory results assessed during screening, in order to permit a quick decision and reduce patient burden. Screening blood samples are nevertheless required to be sent to the central laboratory.

(i) Inclusion Criteria

Each potential subject must satisfy all of the following criteria to be enrolled in the study:

| | |
|---|---|
| 1 | Signed informed consent prior to any study-mandated |
| | procedure |
| 2 | WHO FC II or III. Enrollment will be stratified by |
| | WHO FC II or III. |
| | Proportion of WHO FC II and WHO FC III are expected |
| | to be approximately 40% and 60%, respectively. |
| 3 | PAH etiology belonging to one of the following groups |
| | according to classification (Simmoneau 2019 cited above): |
| | Idiopathic PAH |
| | Heritable PAH |
| | Drugs or toxins induced |
| | PAH associated with connective tissue disease |
| | PAH associated with congenital heart disease, with simple |
| | systemic-to-pulmonary shunt at least 1 year after surgical |
| | repair |
| 4 | First hemodynamic diagnosis of PAH by RHC within |
| | 12 months prior to initiation of selexipag, showing: |
| | mPAP ≥ 25 mmHg and |
| | PAWP or LV end diastolic pressure ≤ 15 mmHg and |
| | PVR > 5 WU (400 dyn · s · cm⁻⁵) and |
| | RVSV ≤ 60 mL as shown in RHC (CO/HR) |
| 5 | Patients already receiving PAH-specific oral mono or dual |
| | therapy (i.e., phosphodiesterase type 5 inhibitors (PDE-5i) or |
| | soluble guanylate cyclase stimulators (sGCs) and/or ERA) or |
| | patients who are not candidates for these therapies. If on |
| | oral PAH-specific therapy, treatment has to be stable (i.e., |
| | no introduction of new therapies or changes in dose) for at |
| | least 90 days prior to both ICF signature and Day 1. |
| 6 | NT-proBNP ≥ 300 ng/L at screening. If local assessment of |
| | BNP (instead of NT-proBNP) is used for eligibility, BNP |
| | measurement of ≥ 50 ng/L will be considered as meeting |
| | inclusion criterion. |
| 7 | Men or women ≥ 18 years (or the legal age of consent in the |
| | jurisdiction in which the study is taking place if greater than |
| | 18) and < 65 years |
| 8 | Women of childbearing potential must: |
| | Have a negative serum pregnancy test during screening and |
| | a negative urine pregnancy test on Day 1, and |
| | Agree to use reliable methods of contraception from Day 1 |
| | to at least 30 days after study drug discontinuation, and |
| | Agree to perform monthly pregnancy tests to at least 30 days |
| | after study drug discontinuation |
| 9 | 6MWD ≥ 150 m during screening period |

(ii) Exclusion Criteria

Any potential subject who meets any of the following criteria will be excluded from participating in the study:

| | |
|---|---|
| 1 | Prior use of IP-receptor agonist, prostacyclin, or |
| | prostacyclin analog. Use of such treatments for |
| | vasoreactivity testing is not exclusionary; intermittent |
| | use of such treatments for digital ulcers or Raynaud's |
| | phenomenon is not exclusionary if stopped > 6 |
| | months (180 days) prior to Day 1 |
| 2 | Treatment with moderate or strong inhibitors of CYP2C8 |
| | (e.g., gemfibrozil, clopidogrel, deferasirox, teriflunomide) |
| | within 28 days prior to Day 1 |
| 3 | Treatment with another investigational drug planned or |
| | taken within 12 weeks (84 days) prior to Day 1 |
| 4 | Cardiopulmonary rehabilitation programs based on |
| | exercise between informed consent and expected |
| | Week 26 visit date |
| 5 | Decompensated cardiac failure requiring hospitalization, |
| | emergency room visit or intravenous diuretics in the |
| | 6 weeks before informed consent |
| 6 | Severe coronary heart disease or unstable angina |
| 7 | Cerebrovascular events (e.g., transient ischemic attack, |
| | stroke) within 3 months prior to Day 1 |
| 8 | Left atrial volume indexed for body surface area ≥ |
| | 43 mL/m², assessed by Echo or cardiac MRI |
| 9 | Myocardial infarction within 6 months prior to Day 1 |
| 10 | Body mass index > 40 kg/m² or body weight < 40 kg |
| 11 | Presence of one or more of the following signs of |
| | relevant lung disease at any time up to Day 1 - if |
| | pulmonary function test results are missing, then |
| | exclusion 11 is considered as met: |
| | Diffusing capacity of the lung for carbon monoxide < |
| | 40% of predicted unless computed tomography |
| | reveals no or mild interstitial lung disease |
| | Forced vital capacity < 60% of predicted. |
| | Forced expiratory volume in 1 second < 60% of |
| | predicted. Pulmonary function tests may be performed |
| | either with or without the use of bronchodilators, as |
| | per local clinical practice. |
| 12 | Known or suspected PVOD |
| 13 | Congenital or acquired valvular defects with clinically |
| | relevant myocardial function disorders not related |
| | to pulmonary hypertension |
| 14 | SBP < 90 mmHg at screening or on Day 1 |
| 15 | Severe renal impairment (estimated creatinine |
| | clearance ≤ 30 mL/min/1.73 m² or serum creatinine > |
| | 2.5 mg/dL at screening) or ongoing or planned dialysis |
| 16 | Known and documented severe hepatic impairment |
| | (with or without cirrhosis) at screening, defined as |
| | Child-Pugh Class C |
| 17 | Known or suspected uncontrolled thyroid disease (hypo- |
| | or hyperthyroidism) |
| 18 | Any hospitalization within 6 weeks prior to informed |
| | consent |
| 19 | Concomitant life-threatening disease with a life |
| | expectancy of less than 12 months |
| 20 | Hemoglobin < 80 g/L at screening |
| 21 | Hypersensitivity to selexipag or any study drug excipient |
| | (mannitol, maize starch, hydroxypropylcellulose, |
| | magnesium stearate, hypromellose, propylene glycol, |
| | titanium dioxide, carnauba wax, iron oxide red, iron oxide |
| | yellow, iron oxide black) |
| 22 | Pregnancy, breastfeeding, or intention to become pregnant |
| | during the study |
| 23 | Any factor or condition likely to affect compliance with |
| | study drug or visit plan |
| 24 | Claustrophobia |
| 25 | MRI-incompatible permanent cardiac pacemaker, |
| | automatic internal cardioverter |
| 26 | Metallic implant (e.g., defibrillator, neurostimulator, |
| | hearing aid, permanent use of infusion device, dental brace, |
| | metal-containing tattoo ink) |
| 27 | Severe arrythmia, atrial fibrillation, multiple premature |
| | ventricular or atrial contractions, or any other condition that |
| | would interfere with proper cardiac gating during MRI |

C. Study Drug

(i) Description of Study Drug

Open-label selexipag will be provided as round, debossed, film-coated tablets in childproof bottles containing 120 tablets.

| | |
|---|---|
| Study drug | Selexipag |
| name | |
| Dosage | Round, film-coated tablets |
| formulation | |
| Unit dose | 200 μg: light-yellow, film-coated tablets |
| strengths/ | with “2” debossed on one side |
| Dosage levels | 400 μg: red, film-coated tablets with “4” |
| | debossed on one side |
| | 600 μg: light-violet, film-coated tablets |
| | with “6” debossed on one side |
| | 800 μg: green, film-coated tablets with |
| | “8” debossed on one side |
| | 1000 μg: orange, film-coated tablets with |
| | “10” debossed on one side |
| | 1200 μg: dark-violet, film-coated tablets |
| | with “12” debossed on one side |
| | 1400 μg: dark-yellow, film-coated tablets |
| | with “14” debossed on one side |
| | 1600 μg: brown, film-coated tablets with |
| | “16” debossed on one side |
| Route of | Oral |
| administration | |
| Dosing | One dose twice daily (in the morning and in |
| instructions | the evening), with or without food, except for |
| | patients with moderate or severe hepatic |
| | impairment (Child-Pugh classes B or C). |
| | For patients with moderate or severe hepatic |
| | impairment (Child-Pugh classes B or C), a once- |
| | daily regimen (one tablet once in the morning |
| | or in the evening) is recommended. |

(ii) Study Drug Administration

The tablets will be administered orally and should be swallowed whole (i.e., not crushed, split, or chewed) with water. On Day 1, the subject will receive only 1 dose, and at each dose change, the first intake of the new dose is to be taken in the evening to reduce the likelihood of the occurrence of prostacyclin-associated AEs. Tolerability may improve when study drug is taken with food. Patients with moderate hepatic impairment (Child-Pugh B) or who are concomitantly taking (a) moderate CYP2C8 inhibitor(s) will start with 200 μg qd in the morning of Day 2.

(ii-a) Study Drug Up-Titration

The goal of the up-titration is to permit each subject to reach his/her personal highest tolerated dose without unmanageable prostacyclin-associated adverse effects. Study drug will be up-titrated to allow each subject to reach their IMD, in the range of 200 to 1600 μg bid. Depending on the dose, a single dose of study drug may consist of 1 or more tablets.

The study drug will be up-titrated from Day 1 to the end of Week 12 (Day 84). Dosing will start at 200 μg twice daily. The site will call the subject once a week from the end of Week 1 to the end of Week 12 and decide whether to increase the dose by 200 μg twice daily (Table 2) if possible. Up-titration will be flexible and may be adapted in case of adverse effects that cannot be relieved with symptomatic treatment, i.e., such as headache, diarrhea, nausea and vomiting, jaw pain, myalgia, pain in extremity, arthralgia, and flushing. In this case, the site may either postpone up-titration by 1 week or down-titrate study drug.

The dose reached at the end of Week 12 will be considered the subject's IMD and will be maintained until EOT.

TABLE 2
Up-Titration Guide

| | | Target selexipag dose | |
|---|---|---|---|
| | Days | (μg, twice per day)^{a} | Comments |
| 1 | 1-7 | 200 | In case of adverse |
| 2 | 8-14 | 400 | effects that cannot be |
| 3 | 15-21 | 600 | relieved with |
| 4 | 22-28 | 800 | symptomatic treatment, |
| 5 | 29-35 | 1000 | postponement of |
| 6 | 36-42 | 1200 | up-titration by 1 week |
| 7 | 43-49 | 1400 | or down-titration |
| 8 | 50-56 | 1600 | should be considered. |
| 9-12 | 57-84 | Buffer period to permit | |
| | | up/down-titration to | |
| | | ensure IMD is reached | |
| | | at the end of Week 12. | |
| 13-52 | 85-364 | IMD | |

^{a}On Day 1, the subject will receive only 1 dose, and at each dose change the first intake of the new dose should be taken in the evening.

(ii-b) Study Drug Maintenance

The maintenance phase will consist of treatment at the subject's IMD from the start at Week 13 to the end of Week 52.

(iii) Study Drug Compliance

Study drug compliance will be based on study drug accountability. Study drug compliance will be calculated by site personnel at each visit using the below formula:

For each strength dispensed:

$$Compliance = [ \frac{( n umber of tablets dispensed - n umber of tablets returned )}{\begin{matrix}total number of tablets that \\ should have been taken during the period\end{matrix}} ] \times 100$$

Between visits, compliance is expected to be between 80% and 120%. Compliance values outside of this range will be considered as a protocol deviation, which will be reported in the clinical trial management system by the clinical research associate.

(iv) Concomitant Therapy

A subject may be enrolled only if therapies received before Day 1 comply with eligibility criteria. From Day 1 to EOT, the following therapies are forbidden and will lead to study drug discontinuation:
Moderate or strong inhibitors of CYP2C8 (e.g., gemfibrozil, clopidogrel, deferasirox, teriflunomide)
Prostacyclin or prostacyclin analog
Any other investigational drug

From Day 1 to completion of EOT assessments, the following therapies may be initiated or up-titrated (if applicable) only if, based on subject's symptoms, that this cannot be postponed to after Week 52 assessments:
Soluble guanylate cyclase stimulator
ERA
PDE-5i
Any other PAH-specific therapy that would become commercially available, provided it is not acting on the prostacyclin pathway

D. Discontinuation of Study Drug and Subject Discontinuation/Withdrawal

(i) Discontinuation of Study Drug

A subject's study drug must be discontinued if:
The subject withdraws consent to receive study drug.
For safety reasons or tolerability reasons, it is in the best interest of the subject to discontinue study drug.
The female subject becomes pregnant.
The subject develops severe hepatic impairment. If hepatic impairment is suspected, a clinical assessment of severity (e.g., Child-Pugh score) must be performed. If a subject has developed severe hepatic impairment (Child-Pugh C) at any time during the study, the study drug must be permanently discontinued.
The subject has pulmonary edema due to PVOD. Should signs of pulmonary edema occur, the possibility of associated PVOD should be considered. If confirmed, the study drug must be discontinued.
Planned initiation of prohibited treatments during the treatment period.
Study drug interruptions exceeding 14 consecutive days.

If a subject discontinues study drug for any reason (except withdrawal of consent from any further participation in the study) before scheduled EOT at Week 52, he/she will continue to attend the visits and assessments as scheduled (i.e., premature EOT and EOS visits), provided the subject's consent for this limited participation in the study has not been withdrawn. If the reason for withdrawal from the study is withdrawal of consent from any further participation in the study, then no additional assessments will be permitted.

(ii) Temporary Interruption of Study Drug

Study drug may be temporarily interrupted in response to an AE, a diagnostic or therapeutic procedure, a laboratory abnormality, or for administrative reasons. Interruptions of study drug must be kept as short as possible. In case of study drug interruption of 3 to 14 days, study treatment should be re-started at a lower dose, and then titrated to the pre-interruption dose. Re-uptitration can be done at scheduled or unscheduled telephone calls or visits.

Study drug interruptions exceeding 14 consecutive days must lead to permanent discontinuation of study drug.

(iii) Subject Discontinuation/Withdrawal from the Study

A subject will not be automatically withdrawn from the study if he/she has to discontinue study drug before the end of the intervention regimen. A subject will be withdrawn from the study for any of the following reasons:
Loss to follow-up
Withdrawal of consent from any further participation in the study
Death
Subject is poorly compliant with study procedures, visits, and assessments, preferably after evaluation and discussion
Continued participation in the study would be contrary to the best interests of the subject.
Sponsor's decision for any reason, including but not limited to premature termination or suspension of the study.

If a subject discontinues study drug and withdraws from the study before the EOS visit for any reason (except for death, loss to follow-up, or withdrawal of consent from any further participation in the study), every attempt should be made to schedule a last appointment/telephone call to assess the safety and well-being of the subject, collect unused study drug, and discuss follow-up medical care.

In the situation where a subject may be at risk for withdrawal of consent and is unable to return for scheduled visits at the protocol-defined frequency, options may be considered for reduced follow-up.

(iv) Loss to Follow-Up

Prior to enrollment, measures should be taken to reduce the chances of a subject being deemed lost to follow-up. These measures include attempts to obtain contact information from each subject, e.g., home, work, and mobile telephone numbers and email addresses for both the subject as well as appropriate family members.

A subject will be considered lost to follow-up if he/she repeatedly fails to return for scheduled visits and is unable to be contacted by the study site. A subject cannot be deemed lost to follow-up until all reasonable efforts made by the study-site personnel to contact the subject are deemed futile.

E. Study Assessments and Procedures

Overview

The schedule of activities (Table 1) summarizes the frequency and timing of efficacy, safety, and exploratory assessments applicable to this study.

For all visits, the subjects must be seen or called on the designated day with an allowed visit window indicated in the schedule of activities. Efficacy assessments at EOT must be performed at trough (i.e., morning dose NOT taken). A follow-up safety telephone call/visit must be performed at least 30 days after intake of the last dose of study drug. If it is not possible to complete all assessments on the same day, a visit may extend over more than 1 day within the allowed time window.

The total blood volume to be collected from each subject will be approximately 69 mL (Table 3).

TABLE 3
Volume of Blood to be Collected From Each Subject

| | | | |
|---|---|---|---|
| | Volume per | No. of | Approximate |
| | Sample | Samples | Total Volume |
| Type of Sample | (mL) | per Subject | of Blood (mL)^{a} |

Safety (including screening and post-intervention assessments)

| | | | |
|---|---|---|---|
| Hematology | 2 | 3 | 6 |
| Clinical chemistry | 6 | 3 | 18 |
| Serum β-hCG | 2.5 | 3 | 7.5 |
| pregnancy tests^{b} | | | |
| NT-proBNP sample | 2.5 | 3 | 7.5 |
| Biomarker samples | 10 | 3 | 30 |
| Approximate total^{c} | 23 | | 69 |

^{a}Calculated as number of samples multiplied by amount of blood per sample; ^{b}Repeated or unscheduled samples may be taken for safety reasons or technical issues with the samples; ^{c}For women of childbearing potential

Unscheduled Visits

Unscheduled visits may be performed at any time during the study. Depending on the reason for the unscheduled visit, appropriate assessments may be performed.

After an unscheduled visit, the regular scheduled study visits must continue according to the planned visit and assessment schedule.

Sample Collection and Handling

See the schedule of activities (Table 1) for the timing and frequency of all sample collections.

(i) Demographics and Baseline Characteristics

Demographic and baseline PAH characteristic data to be collected on all enrolled subjects include: age, sex, race and ethnicity (where local regulations permit), weight and height, date of the initial PAH diagnosis by RHC and WHO FC at screening.

(ii) Efficacy Assessments

(ii-a) Imaging

Image Acquisition: Imaging will be performed as described in Example 1 or in the relevant MRI IAP/Echo IAP. Table 1 provides the timing for MRI acquisition.

(ii-b) 6MWT: Exercise capacity will be measured by the 6MWT. The 6MWT is a non-encouraged test that measures the distance walked in 6 minutes. At Week 26 and EOT visit, subjects must NOT take the morning study drug dose before any visit-related procedure. Dyspnea will be assessed by the BDI CR 10 Scale®, a scale used to quantify the degree of shortness of breath before and at the end of the 6MWT.

(ii-c) WHO FC: WHO FC will be determined as per WHO definition.

(ii-d) Risk Stratification: Clinical signs of right heart failure, progression of PAH symptoms, and syncope will be collected at screening and Week 26 visit; and will be used for risk stratification.

(ii-e) NT-proBNP: A blood sample will be drawn for the analysis of NT-proBNP. NT-proBNP results will be assessed by the central laboratory. Details regarding blood sampling procedures, collection, and shipment of the samples will be described in the laboratory manual.

(ii-f) Biomarkers: It is hypothesized that selexipag may have a beneficial effect on circulating biomarkers involved in RV function and structure; therefore, changes in such biomarkers from baseline to Week 26 and Week 52 will be explored. Biomarkers will be measured after the EOS and will be based on the latest scientific evidence regarding RV function and structure at the time of laboratory analysis. No genetic testing of any kind will be performed.

Serum samples will be stored. They will be used to assess additional exploratory biomarkers after the EOS. The exploratory biomarkers will be based on the latest scientific evidence regarding RV function and structure at the time of laboratory analysis.

(iii) Safety Assessments

The standard assessments to evaluate the safety and tolerability of selexipag in this study include reporting and follow-up of (S)AEs, pregnancies, physical examination, vital signs, and safety laboratory tests until the EOS visit (Table 1).

Any clinically significant abnormalities persisting at the EOS/early withdrawal will be followed until resolution or until a clinically stable condition is reached or until the subject has been deemed lost to follow-up.

(iii-a) Physical Examination

Physical examination will include the evaluation of the subject's height (only at screening), the general appearance, heart and lungs. Other examinations will be performed if indicated, based on medical history and/or symptoms. Height will be measured without shoes.

(iii-b) Vital Signs

Heart rate (HR), systolic blood pressure (SBP), diastolic blood pressure (DBP) and body weight, will be assessed during on-site visits.

Blood pressure and pulse/heart rate measurements will be assessed in a supine or sitting position with an automated device. Manual techniques will be used only if an automated device is not available. It is recommended that the patient is allowed to rest for at least 5 minutes before the measurement. It is also recommended that measurements are performed on the same arm and in the same position (supine or sitting) throughout the study for each individual patient. Vital signs are to be measured prior to blood collection.

Body weight will be measured in indoor clothing without shoes.

(iii-c) Clinical Safety Laboratory Assessments

Blood samples for clinical chemistry and hematology will be collected as described herein. Safety laboratory analyses will include:
Basic hematology panel
Clinical chemistry panel including TSH, T3, and T4
Pregnancy tests for females of childbearing potential. A serum pregnancy test will be performed at the site at screening, at Week 26 and at Week 52 (EOT); urine pregnancy tests will be performed at the site on Day 1, and at the subject's home monthly between Day 1 and EOS, except for Month 6 and Month 12, when a serum pregnancy test is performed at the site visit (Weeks 26 and 52).
Any clinically relevant laboratory abnormalities detected after signing of informed consent must be reported as an AE or SAE as appropriate.

(iv) Adverse Events and Serious Adverse Events

AEs will be reported by the subject for the duration of the study (or, when appropriate, by a caregiver, surrogate, or the patient's legally acceptable representative).

(iv-a) Time Period and Frequency for Collecting Adverse Event and Serious Adverse Event Information

Serious Adverse Events: Information regarding SAEs, as well as PQC, must be transmitted within 24 hours.

(iv-b) Pregnancy

All initial reports of pregnancy in female subjects or partners or male patients must be reported by the study-site personnel within 24 hours of their knowledge of the event using the appropriate Pregnancy Notification Form. Abnormal pregnancy outcomes (e.g., spontaneous abortion, fetal death, stillbirth, congenital anomalies, ectopic pregnancy) are considered SAEs and must be reported using an SAE reporting form. Any subject who becomes pregnant during the study must discontinue further study drug. Follow-up information regarding the outcome of the pregnancy and any postnatal sequelae in the infant will be required. The duration of this follow-up will depend on the clinical conditions of the newborn and may be extended until discharge from hospital when applicable.

(v) Patient's Experience

Patient experience will be assessed with the 3-level version of EQ-5D (EQ-5D-3L) questionnaire during Day 1, at Week 26, and at Week 52 (EOT) at the site.

The EQ-5D-3L questionnaire has 2 components: health state description and visual analogue scale (EQ VAS) for patient's self-rating. In the description part, health status is measured in 5 dimensions: mobility, self-care, usual activities, pain/discomfort, and anxiety/depression at 3 levels: no problems, some problems, and extreme problems.

F. Statistical Considerations

A general description of the statistical methods to be used to analyze the efficacy and safety data is outlined below.

(i) Statistical Hypotheses

The null statistical hypothesis is that the mean change from baseline to Week 26 in RVSV is equal to zero. The alternative statistical hypothesis is that the mean change from baseline in RVSV is different from zero.

(ii) Sample Size Determination

Sample size calculation for the primary endpoint is based on the following assumptions:
A 2-sided Type I error of 5% and a Type II error of 10% (90% power)
A mean change from baseline to Week 26 in RVSV of +8 mL
A standard deviation (SD) of 20 mL for the change from baseline to Week 26 in RVSV
A normal distribution for the change from baseline to Week 26 in RVSV
15% of subjects with non-evaluable RVSV assessment at baseline and/or postbaseline

Based on the above assumptions, a total of 80 subjects (68 analyzable+12 to account for 15% non-evaluable) must be enrolled in order to establish whether change from baseline to Week 26 in RVSV is different from zero (based on paired t-test for single arm).

The assumption that the change from baseline to Week 26 in RVSV will be +8 mL is clinically relevant, i.e., a difference of 8 mL to 12 mL is clinically relevant.

Table 4 displays power calculations for different assumptions of treatment effect and associated SD, given a fixed number of 68 analyzable subjects (80 in total).

TABLE 4
Sample Size Power Sensitivity

| | Change from | | |
|---|---|---|---|
| | baseline to Week | Associated | |
| Scenario | 26 in RVSV (mL) | SD | Power |
| Retained scenario | 8 | 20 | 90% |
| Sensitivity 1 | 8 | 22 | 84% |
| Sensitivity 2 | 8 | 25 | 74% |
| Sensitivity 3 | 10 | 20 | 98% |
| Sensitivity 4 | 10 | 22 | 96% |
| Sensitivity 5 | 10 | 25 | 90% |

(iii) Populations for Analyses

For purposes of analysis, the following populations are defined:
Screened Analysis Set: The Screened Analysis Set will include all subjects who were screened and received a subject number.
Safety Set: The SS will include all subjects from the Screened Analysis Set who received at least 1 dose of study drug.
Full Analysis Set: The FAS will include all subjects from the SS who had a baseline as well as a postbaseline measurement for RVSV assessed by cardiac MRI from pulmonary artery flow.
Per-protocol Analysis Set: The PPS will include all subjects in the FAS without major protocol deviations that could affect the main analysis of the primary efficacy variable.

(iv) Statistical Analyses

(iv-a) General Considerations

The overall type I error is α=0.05. All analyses will be performed using 2-sided statistical tests. If the primary endpoint analysis is statistically significant (i.e., p-value below nominal alpha), the study will be declared positive. No multiplicity adjustments will be performed for secondary/exploratory endpoints, and therefore, all associated p-values are of exploratory nature. No interim analysis will be performed. Subjects without a baseline value will be excluded from the corresponding analysis. Subgroup analyses on primary and secondary endpoints will include WHO FC (II/III) at baseline. All subgroup analyses will be of exploratory nature.

(iv-b) Primary Endpoint

The primary efficacy analysis will be performed on the FAS. RVSV will be assessed by pulmonary artery flow MRI and summarized by timepoint (baseline and Week 26) using descriptive statistics (n, mean, SD, median, Q1 and Q3). The change from baseline to Week 26 in RVSV will be summarized similarly. Change from baseline in RVSV will be analyzed at α=0.05 (2-sided) using an ANCOVA with a factor for WHO FC (II/III) at baseline and a covariate for baseline RVSV. The mean change from baseline and 95% CI will be estimated based on the model. A sensitivity analysis will be performed on the PPS. Another sensitivity analysis will be performed on the SS, where subjects with missing postbaseline RVSV will be imputed using their baseline value.

(iv-c) Secondary Endpoints

The secondary endpoints are to (i) assess the effects of selexipag on disease severity and exercise capacity in patients with PAH, (ii) evaluate the safety and tolerability of selexipag in patients with PAH, and (iii) evaluate the effect of selexipag on risk stratification in PAH.

Change from baseline to Week 26 in RVEDV, RVESV, RVEF, RV mass, RVGLS, 6MWD, and number of low-risk criteria will be summarized descriptively by timepoint and analyzed at α=0.05 (2-sided) on the FAS using an ANCOVA with a factor for WHO FC (II/III) at baseline and a covariate for baseline value. The mean change from baseline and 95% CI will be estimated based on the model.

WHO FC will be summarized on the FAS by timepoint using frequency tables. Changes from baseline in WHO FC will be dichotomized as worsening (i.e., change >0) versus no change or improvement (i.e., change ≤0). Worsening will be analyzed at α=0.05 (2-sided) using a logistic regression model with a factor for WHO FC (II/III) at baseline.

NT-proBNP will be summarized on the FAS by timepoint using descriptive statistics as well as geometric means and CVs. The Week 26 versus baseline ratio will be summarized similarly. The ratio versus baseline in NT-proBNP will be log-transformed and analyzed at α=0.05 (2-sided) using an ANCOVA with a factor for WHO FC (II/III) at baseline and a covariate for baseline log NT-proBNP.

A sensitivity analysis will be performed on the SS for all secondary endpoints, where patients with missing post-baseline value will be imputed using their baseline value.

All analyses for secondary endpoints are of exploratory nature because there will be no adjustment for multiplicity. Additional analyses will be performed on the SS for all secondary endpoints, where subjects with missing postbaseline value will be imputed using their baseline value.

(iv-d) Exploratory Endpoints

Exploratory variables will be analyzed at α=0.05 (2-sided) on the FAS and SS.

(iv-e) Safety Analyses

Safety analyses will be performed on the SS.

Adverse Events

A treatment-emergent AE is any AE from first dose up to 3 days after end of study drug. The number and percentage of subjects experiencing at least 1 treatment-emergent AE or SAE will be tabulated by:

Medical Dictionary for Regulatory Activities system organ class and individual preferred term, in descending order of incidence.

Frequency of subjects with events coded with the same preferred term, in descending order of incidence.

Furthermore, treatment-emergent AEs and SAEs will be tabulated as described above by severity and relationship to study drug. SAEs will be also tabulated up to 30 days after end of study drug. AEs leading to premature discontinuation of study drug, AEs with an outcome of death, and AEs of special interest (AESi) will be summarized as described above. AEs occurring during titration period will also be summarized separately. Listings will be provided for all reported AEs, including SAEs. In addition, separate listings, as well as narratives, will be provided for SAEs, for AEs leading to premature discontinuation of study drug, and for AEs with an outcome of death.

The following AEs will be listed only:
AEs occurring prior to first study drug dose
Non-serious AEs occurring after EOT+3 days
SAEs occurring more than 30 days after discontinuation of study drug

Clinical Laboratory Tests

Descriptive summary statistics by visit will be provided for observed values and absolute changes from baseline, in both hematology and clinical chemistry laboratory tests. In order to minimize missing data and to allow for out-of-window visits, all recorded assessments up to EOT+3 days will be assigned to the most appropriate visit timepoint according to the best fitting time window for that assessment.

Marked laboratory abnormalities will be summarized for each laboratory variable providing their incidence and frequency. Absolute values and changes from baseline of laboratory values during the course of the study will also be summarized. The number and percentage of subjects with treatment-emergent laboratory abnormalities will be tabulated.

The following tests will be performed:

| Laboratory | | |
|---|---|---|
| Assessments | Parameters | |
| Hematology | Platelet count | WBC count with differential: |
| | RBC count | Neutrophils |
| | Hemoglobin | Lymphocytes |
| | Hematocrit | Monocytes |
| | MCV | Eosinophils |
| | RBC morphology | Basophils |

| | |
|---|---|
| | A WBC evaluation may include any abnormal cells, |
| | which will then be reported by the laboratory. An |
| | RBC evaluation may include abnormalities in the |
| | RBC count, RBC parameters, or RBC morphology, |
| | which will then be reported by the laboratory. In |
| | addition, any other abnormal cells in a blood |
| | smear will also be reported. |
| Clinical | Sodium |
| Chemistry | Potassium |
| | Glomerular filtration rate, using the Modification |
| | of Diet in Renal Disease formula |
| | Creatinine |
| | AST |
| | ALT |
| | Total and direct bilirubin |
| | Alkaline phosphatase |
| | Thyroid hormones: |
| | Free and total triiodothyronine (T3) |
| | Free and total thyroxine (T4) |
| | Thyroid stimulating hormone (TSH) |
| NT-proBNP | NT-proBNP |
| Pregnancy | Serum and urine pregnancy testing for women |
| | of childbearing potential only |
| Biomarkers | Exploratory biomarkers to be measured after |
| | the end of the study will be based on the latest |
| | scientific evidence regarding RV function and |
| | structure at the time of laboratory analysis. No |
| | genetic testing of any kind will be performed. |

G. Adverse Events

(i) Definitions and Classifications

Adverse Event

An adverse event is any untoward medical occurrence in a clinical study subject administered a medicinal (investigational or non-investigational) product. An adverse event does not necessarily have a causal relationship with the intervention. An adverse event can therefore be any unfavorable and unintended sign (including an abnormal finding), symptom, or disease temporally associated with the use of a medicinal (investigational or non-investigational) product, whether or not related to that medicinal (investigational or non-investigational) product. (Definition per ICH). This includes any occurrence that is new in onset or aggravated in severity or frequency from the baseline condition, or abnormal results of diagnostic procedures, including laboratory test abnormalities.

Serious Adverse Event

A serious adverse event based on ICH and European Union Guidelines on Pharmacovigilance for Medicinal Products for Human Use is any untoward medical occurrence that at any dose:
Results in death
Is life-threatening (The subject was at risk of death at the time of the event. It does not refer to an event that hypothetically might have caused death if it were more severe.)
Requires inpatient hospitalization or prolongation of existing hospitalization
Results in persistent or significant disability/incapacity
Is a congenital anomaly/birth defect
Is a suspected transmission of any infectious agent via a medicinal product
Is Medically Important

If a serious and unexpected adverse event occurs for which there is evidence suggesting a causal relationship between the study drug and the event, the event must be reported as a serious and unexpected suspected adverse reaction even if it is a component of the study endpoint.

Unlisted (Unexpected) Adverse Event/Reference Safety Information

An adverse event is considered unlisted if the nature or severity is not consistent with the applicable product reference safety information. For selexipag, the expectedness of an AE will be determined by whether or not it is listed in the IB

(ii) Attribution Definitions

Assessment of Causality: The causal relationship to study drug is determined by the Investigator. The following selection should be used to assess all AEs.

Related: There is a reasonable causal relationship between study drug administration and the AE.

Not Related: There is not a reasonable causal relationship between study drug administration and the AE. The term “reasonable causal relationship” means there is evidence to support a causal relationship.

(iii) Severity Criteria

An assessment of severity grade will be made using the following general categorical descriptors:

Mild: Awareness of symptoms that are easily tolerated, causing minimal discomfort and not interfering with everyday activities.

Moderate: Sufficient discomfort is present to cause interference with normal activity.

Severe: Extreme distress, causing significant impairment of functioning or incapacitation. Prevents normal everyday activities.

(iv) Procedures

All Adverse Events

Whenever possible, diagnoses should be given when signs and symptoms are due to a common etiology (e.g., cough, runny nose, sneezing, sore throat, and head congestion should be reported as “upper respiratory infection”).

Serious Adverse Events

All SAEs that have not resolved at time of the EOS visit must be followed until any of the following occurs:
The event resolves
The event stabilizes
The event returns to baseline, if a baseline value/status is available
The event can be attributed to agents other than the study drug or to factors unrelated to study conduct
It becomes unlikely that any additional information can be obtained (patient or health care practitioner refusal to provide additional information, lost to follow-up)

Any event requiring hospitalization (or prolongation of hospitalization) that occurs during participation in the study must be reported as an SAE, except hospitalizations for the following:
Hospitalizations not intended to treat an acute illness or AE (e.g., social reasons such as pending placement in long-term care facility, cosmetic elective surgery).
Surgery or procedure planned before entry into the study or standard monitoring of a pre-existing disease or medical condition that did not worsen, e.g., hospitalization for coronary angiography in a patient with stable angina pectoris. Hospitalizations that were planned before the signing of the ICF, and where the underlying condition for which the hospitalization was planned has not worsened, will not be considered SAEs. Any AE that results in a prolongation of the originally planned hospitalization is to be reported as a new SAE.
For convenience the investigator may choose to hospitalize the patient for the duration of the intervention period.

If the patient is hospitalized in a hospital other than the study site, it is the investigator's responsibility to contact this hospital to obtain all SAE-relevant information and documentation.

The cause of death of a patient in a study within 30 days of the last dose of study drug, whether or not the event is expected or associated with the study drug, is considered an SAE.

Information regarding SAEs will be transmitted within 24 hours.

Follow-Up of Serious Adverse Events

All serious adverse events that have not resolved by the end of the study, or that have not resolved upon discontinuation of the subject's participation in the study, must be followed until any of the following occurs:
The event resolves
The event stabilizes
The event returns to baseline, if a baseline value/status is available
The event can be attributed to agents other than the study drug or to factors unrelated to study conduct
It becomes unlikely that any additional information can be obtained (subject or health care practitioner refusal to provide additional information, lost to follow-up after demonstration of due diligence with follow-up efforts)
Suspected transmission of an infectious agent by a medicinal product will be reported as a serious adverse event. Any event requiring hospitalization (or prolongation of hospitalization) that occurs during the course of a subject's participation in a study must be reported as a serious adverse event, except hospitalizations for the following:
Hospitalizations not intended to treat an acute illness or adverse event (e.g., social reasons such as pending placement in long-term care facility).
Surgery or procedure planned before entry into the study. Hospitalizations that were planned before the study, and where the underlying condition for which the hospitalization was planned has not worsened, will not be considered serious adverse events. Any adverse event that results in a prolongation of the originally planned hospitalization is to be reported as a new serious adverse event.
For convenience the subject may be hospitalized for a study visit.

The cause of death of a subject in a study within 30 days of the last dose of study drug, whether or not the event is expected or associated with the study drug, is considered a serious adverse event.

1-27. (canceled)

28. A method for treating pulmonary arterial hypertension in a patient in need thereof, comprising:
(a) performing magnetic resonance imaging (MRI) on the right ventricle of the patient to provide a MRI baseline image;
(b) administering a therapeutically effective amount of selexipag to the patient;
(c) performing MRI on the right ventricle of the patient to provide a MRI test image; and
(d) comparing the MRI baseline image with the MRI test image.

29. The method of claim 28, wherein step (d) is performed at least about 26 weeks or at least about 52 weeks after initiating the administration of the selexipag.

30. The method of claim 28, wherein step (d) is performed at about 26 weeks and at about 52 weeks after initiating the administration of selexipag.

31. The method of claim 28, wherein the treatment is adjusted based on step (d).

32. The method of claim 28, wherein, prior to initiating the administration of the selexipag, the patient is in World Health Organization functional class II or III or has a NT-proBNP >300 ng/L prior to the initiation of the administration of the selexipag.

33. The method of claim 28, wherein the patient has not received treatment using an IP-receptor agonist, prostacyclin, or prostacyclin analog within at least 6 months prior to the initiation of the administration of the selexipag.

34. The method of claim 28, wherein the selexipag is administered at a starting dose and is increased to determine an individual maximum tolerated dose (iMTD).

35. The method of claim 34, wherein the starting dose is about 200 μg twice daily.

36. The method of claim 34, wherein the iMTD is from about 200 μg to about 1600 μg twice daily.

37. The method of claim 34, wherein the iMTD does not exceed about 1600 μg twice daily.

38. The method of claim 34, wherein the iMTD is maintained during a maintenance phase following a dose adjustment phase.

39. The method of claim 28, wherein the patient receives background therapy comprising phosphodiesterase type 5 inhibitors, soluble guanylate cyclase stimulators, or endothelin receptor agonist, wherein such therapy is present for at least three months at a stable dose prior to administration of selexipag.

40. The method of claim 28, wherein the pulmonary arterial hypertension is idiopathic pulmonary arterial hypertension, heritable pulmonary arterial hypertension, drugs or toxins induced, pulmonary arterial hypertension associated with connective tissue disease, or pulmonary arterial hypertension associated with congenital heart disease, with simple systemic-to-pulmonary shunt at least 1 year after surgical repair.

41. The method of claim 28, wherein the patient's right ventricular stroke volume (RVSV), as measured by MRI, increases after about 26 weeks or about 52 weeks following the selexipag initiation.

42. The method of claim 41, wherein the increase is from about 8 mL to about 12 mL.

43. The method of claim 28, wherein the patient's right ventricular end diastolic volume (RVEDV), as measured by MRI, decreases after about 26 weeks or about 52 weeks following the selexipag initiation.

44. The method of claim 28, wherein the patient's right ventricular end systolic volume (RVESV), as measured by MRI, decreases after about 26 weeks or about 52 weeks following the selexipag initiation.

45. The method of claim 28, wherein the patient's right ventricular ejection fraction (RVEF), as measured by MRI, increases after about 26 weeks or about 52 weeks following the selexipag initiation.

46. The method of claim 28, wherein the patient's right ventricular (RV) mass, as measured by MRI, decreases after about 12 months following the selexipag initiation.

47. The method of claim 28, wherein the patient's right ventricular global longitudinal strain (RVGLS), as measured by MRI, improves after about 26 weeks or about 52 weeks following the selexipag initiation.

48. The method of claim 28, wherein the patient's right main PA pulsatility, as measured by MRI, improves after about 26 weeks or after about 52 weeks following selexipag initiation.

49. The method of claim 28, wherein the selexipag is inhaled, orally administered, or parenterally administered.

50. The method of claim 49, wherein the selexipag is inhaled.

51. The method of claim 49, wherein the selexipag is parenterally administered.

52. The method of claim 51, wherein the parenteral administration is subcutaneous or intravenous.

53. The method of claim 49, wherein the selexipag is an orally administered.

54. The method of claim 53, wherein the selexipag is the form of a tablet.
